(19) United States
(12) Patent Application Publication
(10) Pub. No.: US 2026/0042999 A1
(43) Pub. Date: Feb. 12, 2026

(54) NON-ANTIBIOTIC ANTIMICROBIAL COMPOSITIONS

(71) Applicant: Lant Medical Ltd., Alnmouth Northumberland (GB)

(72) Inventor: Joanne LANT, Alnmouth Northumberland (GB)

(73) Assignee: Lant Medical Ltd., Alnmouth Northumberland (GB)

(21) Appl. No.: 19/110,236

(22) PCT Filed: Mar. 28, 2023
(86) PCT No.: PCT/GB2023/050798

§ 371 Date: Mar. 10, 2025

(30) Foreign Application Priority Data
Mar. 28, 2022 (GB) 2204335.0

(51) Int. Cl. C12N 1/20; A61K 31/191; A61K 31/194; A61K 31/355; A61K 31/59; A61K 31/722; A61K 33/06; A61K 33/10; A61K 35/00; A61P 1/04; A61P 31/04

(52) U.S. Cl. CPC C12N 1/20; A61K 31/191; A61K 31/194; A61K 31/355; A61K 31/59; A61K 31/722; A61K 33/06; A61K 33/10; A61P 1/04; A61P 31/04; A61K 2035/11

(57) ABSTRACT

The present invention relates to a non-antibiotic antimicrobial composition, new uses and methods of medical treatment or prophylaxis. In particular, the present invention relates to a non-antibiotic antimicrobial composition comprising a zeolite and at least one member of the Lactobacilli genus for the treatment of a disease or a condition associated with or caused by Helicobacter spp such as Helicobacter pylori.

### CLAIM FOR PRIORITY

This application is a national phase application of PCT/GB2023/050798 filed March 2023. The priorities of PCT/GB2023/050798 are hereby claimed and their disclosures incorporated herein by reference.

### BACKGROUND OF THE INVENTION

The present invention relates to a non-antibiotic antimicrobial composition, new uses and methods of medical treatment or prophylaxis. In particular, the present invention relates to a non-antibiotic antimicrobial composition comprising a zeolite and at least one member of the Lactobacilli genus for the treatment of a disease or a condition associated with or caused by Helicobacter spp such as Helicobacter pylori. The invention further relates to a non-antibiotic antimicrobial composition comprising a zeolite and at least one member of the Lactobacilli genus for the treatment of an infection with a urease active bacteria in the gastro-intestinal tract.

### INTRODUCTION

Infectious diseases including bacterial infections continue to be a serious health problem worldwide. The Gram negative (−ve) microorganism Helicobacter pylori was first cultivated in 1982 and since then it has become apparent that many related species can often be found in humans and other animals. These Helicobacter species (spp) are generally classified according to their ability to invade and colonize gastric or enterohepatic tissues. Colonization of gastric mucosa appears to be most prevalent in the surface mucus layer and within the gastric glands and parietal cells.

Gastric Helicobacter spp are widely distributed in mammalian hosts and are many cases could cause an inflammatory response resembling that seen with H. pylori in humans. Similarly, enterohepatic Helicobacter spp are also a diverse group of microorganisms which can be found in the intestinal tract and the liver of humans, other mammals, and birds. They are frequently linked with inflammation or malignant transformation in immunocompetent hosts and with more severe clinical disease in immunocompromised humans and animals.

The primary disorder, which occurs after colonisation with H. pylori, is chronic active gastritis. This condition can be observed in all H. pylori-positive subjects. The intragastric distribution and severity of this chronic inflammatory process depend on a variety of factors, such as characteristics of the colonizing strain, host genetics and immune response, diet, and the level of acid production. H. pylori-induced ulcer disease, gastric cancer, and lymphoma are all complications of this chronic inflammation; ulcer disease and gastric cancer in particular occur in those individuals and at those sites with the most severe inflammation (Kusters, Johannes G et al., “Pathogenesis of Helicobacter pylori infection.” Clinical microbiology reviews vol. 19,3 (2006):449-90. doi:10.1128/CMR.00054-05).

More than 50% of the world population is infected with this stomach bacterium. Severity of the infection is associated with bacterial load (Celli, Jonathan P et al. “Helicobacter pylori moves through mucus by reducing mucin viscoelasticity.” Proceedings of the National Academy of Sciences of the United States of America vol. 106, 34 (2009):14321-6. doi:10.1073/pnas.0903438106; Varbanova M, Malfertheiner P. Bacterial load and degree of gastric mucosal inflammation in Helicobacter pylori infection. Dig Dis. 2011; 29:592-599. doi:10.1159/000333260).

Treatment of Helicobacter pylori infection remains a challenge with auxiliary detrimental effects. The standard triple therapy (STT), which consists of the association of a proton pump inhibitor and two antibiotics, one of which most commonly includes clarithromycin as a key agent, has been used empirically worldwide for two decades without antimicrobial susceptibility testing (AST) as first-line therapy. Since 2010, a marked decrease in efficacy of this treatment regimen has been observed globally, which resulted in the recommendation to prescribe quadruple therapies with or without bismuth when clarithromycin resistance was over 15% (Maastricht V) (Malfertheiner P, Megraud F, O'Morain C A, et al., Management of Helicobacter pylori infection-the Maastricht V/Florence consensus report. Gut 2017; 66:6-30. doi:10.1136/gutjnl-2016-312288 pmid:http://www.ncbi.nlm.nih.gov/pubmed/27707777).

These regimens have inherent drawbacks that may promote further increases in antimicrobial resistance and induce gut microbiota dysbiosis because of the empiric use of multiple antibiotics (Suzuki S. a·Kusano C. b·Horii T. a·Ichijima R. a·Ikehara H. a et al. The Ideal Helicobacter pylori Treatment for the Present and the Future. Digestion 2022; 103:62-68 https://doi.org/10.1159/000519413). With antibiotic resistance identified as one of the top 10 threats to global public health by the World Health Organisation.

H. pylori resistance to antibiotics has been monitored at the European level every 10 years, beginning in 1998 (Glupczynski Y, Méraud F, Lopez-Brea M, et al., European multicentre survey of in vitro antimicrobial resistance in Helicobacter pylori. Eur J Clin Microbiol Infect Dis 2001; 20:820-823. doi:10.1007/s100960100611 pmid: http: //www. ncbi. nlm. nih. gov/pubmed/11783701), 2008 (Megraud F, Coenen S, Versporten A, et al., Helicobacter pylori resistance to antibiotics in Europe and its relationship to antibiotic consumption. Gut 2013; 62:34-42. doi:10.1136/gutjnl-2012- 302254) and then again in 2018 (Megraud F, Bruyndonckx R, Coenen S The European Helicobacter pylori Antimicrobial Susceptibility Testing Working Group, et al., Helicobacter pylori resistance to antibiotics in Europe in 2018 and its relationship to antibiotic consumption in the community. Gut 2021; 70:1815-1822).

In the last study in 2018 a positive correlation between H. pylori resistance and the consumption of the corresponding antibiotics in the community was confirmed. The patients'mean age was 51.2 years (range 17-91 years). The distribution by age was in line with the usual distribution observed among upper digestive endoscopy patients. It is thought many people are unaware they carry H. pylori, until later in life. Unless treated effectively, an infection by H. pylori would usually persist indefinitely, manifesting as other seemingly unrelated disorders and diseases.

Despite the efforts to find effective treatments against Helicobacter spp., reports on specific and effective therapies with low resistance and desired efficiency are rare if not absent.

Thus, currently, there are no effective non-antibiotic treatments against the Helicobacte rspp. There are also no efficacious treatments which can be widespread and accepted as a long-term user-friendly treatment standards of Helicobacter spp., absent the associated detrimental effects described above.

Therefore, there is a pressing need for developing not only effective treatments against Helicobacter spp. but also treatment which can be widely adopted, lack toxicity and avoid the unfettered reliance on antibiotics.

H. Pylori is a urease active bacteria. Other urease positive bacteria have also been identified as pathogenic in the gastro-intestinal tract. Staphylococcus capitius urealiticum causes urinary tract infections, as do members of the Proteus spp. family such as Proteus mirabilis. Klebsiella spp. such as Klebsiella pneumoniae is associated with pneumonia in the gastrointestinal tract. Mycobacterium such as Mycobacterium tuberculosis is associated with intestinal tuberculosis. Therefore there is also a need for effective treatments against infection with pathogenic urease positive bacteria which again can be widely adopted, lack toxicity and avoid the unfettered reliance on antibiotics.

### SUMMARY

The present invention relates to a non-antibiotic antimicrobial composition, new uses and methods of medical treatment or prophylaxis.

Specifically, the present invention relates to a non-antibiotic antimicrobial composition comprising a zeolite and at least one member of the Lactobacilli genus for the treatment of a condition or disease associated with or caused by Helicobacter spp in vertebrates.

Specifically, the present invention further relates to a non-antibiotic antimicrobial composition comprising a zeolite and at least one member of the Lactobacilli genus for the treatment of a condition or disease associated with or caused by pathogenic urease active bacteria in vertebrates.

The pathogenic urease active bacteria in vertebrates may be one or more taken from the list of Helicobacter spp., Staphylococcus spp., Proteus spp., Klebsiella spp., and Mycobacterium spp.

The pathogenic urease active bacteria in vertebrates may be one or more taken from the list of Helicobacter pylori, Staphylococcus capitius urealiticum, Proteus mirabilis, Klebsiella pneumoniae, and Mycobacterium tuberculosis.

In one aspect, there is provided an antimicrobial composition comprising a zeolite and at least one member of the Lactobacilli genus, for use in the treatment of medical conditions associated with Helicobacter, in vertebrates.

In another aspect there is provided an antimicrobial composition comprising a zeolite and at least one member of the Lactobacilli genus, for use in the treatment of medical conditions associated with or caused by pathogenic urease active bacteria in vertebrates. The pathogenic urease active bacteria in vertebrates may be one or more taken from the list of Helicobacter spp, Staphylococcus spp., Proteus spp., Klebsiella spp., and Mycobacterium spp. More specifically, The pathogenic urease active bacteria in vertebrates may be one or more taken from the list of Helicobacter pylorus, Staphylococcus capitius urealiticum, Proteus mirabilis, Klebsiella pneumoniae, and Mycobacterium tuberculosis.

In some embodiments, there is provided an antimicrobial composition wherein the zeolite is naturally occurring or synthetic.

In some embodiments, there is provided an antimicrobial composition wherein the zeolite is zeolite clinoptilolite (ZC).

In some embodiments, there is provided an antimicrobial composition wherein the zeolite is activated zeolite clinoptilolite (aZC), preferably double activated zeolite clinoptilolite (aZC).

In some embodiments, there is provided an antimicrobial composition wherein the at least one member of the Lactobacilli genus is selected from the group consisting of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii.

In some embodiments, there is provided an antimicrobial composition wherein the at least one member of the Lactobacilli genus is Lactobacillus reuteri.

In some embodiments, there is provided an antimicrobial composition wherein the Lactobacillus reuteri is Lactobacillus reuteri DSM17648 or Lactobacillus reuteri UBLRu-87.

In some embodiments, there is provided an antimicrobial composition wherein the vertebrate is administered with a therapeutically effective amount of the antimicrobial composition.

In some embodiments, there is provided an antimicrobial composition wherein the therapeutically effective amount is achieved by a regimen of administration of the zeolite and the at least one member of the Lactobacilli genus.

In some embodiments, there is provided an antimicrobial composition wherein the regimen comprises one or more types of administration of the zeolite and the at least one member of the Lactobacilli genus. In some embodiments, the regimen of administration of the antimicrobial composition is suitable for peroral, rectal, topical, enteral, vaginal, buccal, orthotopic, intratracheal, intralesional, endoscopical, transmucosal, sublingual, intestinal administration and combinations thereof. In some embodiments, the regimen of administration of the antimicrobial composition is suitable for peroral.

In some embodiments, there is provided an antimicrobial wherein the at least one member of the Lactobacilli genus is viable.

In some embodiments, there is provided an antimicrobial composition wherein the antimicrobial composition further comprises biologically active substances or minerals such as calcium carbonate, magnesium carbonate, vitamins, such as D and E, pharmaceutically acceptable carriers, additives and adjuvants, as well as antimicrobial chitin, such as chitosan and alpha-ketoglutarate, citrate and lactate.

In some embodiments, there is provided an antimicrobial composition wherein the regimen of administration of the zeolite and the at least one member of the Lactobacilli genus, is suitable for peroral administration or gastric administration.

In some embodiments, there is provided an antimicrobial composition wherein the composition comprises, activated zeolite clinoptilolite (aZC), Lactobacillus reuteri DSM17648 or Lactobacillus reuteri UBLRu-87, calcium carbonate and magnesium carbonate.

In some embodiments, there is provided an antimicrobial composition wherein the medical condition is associated with Helicobacter pylori.

In some embodiments, there is provided an antimicrobial wherein the vertebrate is a human.

In some embodiments, there is provided an antimicrobial composition wherein the antimicrobial composition leads to a reduction of one or more markers.

In some embodiments, there is provided an antimicrobial composition wherein the marker is a predictive marker selected from the group consisting of urea, gastric ammonia and urease activity.

In some embodiments, there is provided an antimicrobial composition wherein the predictive marker is gastric ammonia.

In some embodiments, there is provided an antimicrobial composition wherein the antimicrobial composition leads to reduction of gastric ammonia levels in a vertebrate infected by Helicobacter pylori.

In some embodiments, there is provided an antimicrobial composition wherein the reduction of gastric ammonia is at least 5%, 10%, 15%, 20%, 25%, 30%, 35%, 40%, 45%, 50%, 55%, 60%, 65%, 70%, 75%, 80%, 85%, 90%, 95% or 100% or greater compared to placebo.

In some embodiments, there is provided an antimicrobial composition wherein the reduction of gastric ammonia is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to placebo.

In some embodiments, there is provided an antimicrobial composition wherein the reduction of the predictive marker is statistically significant compared to a placebo.

In some embodiments, there is provided an antimicrobial composition wherein the reduction of gastric ammonia marker levels in a human infected with a pathogenic urease active bacteria is statistically significant.

In some embodiments, there is provided an antimicrobial composition wherein the reduction of gastric ammonia marker levels in a human infected with Helicobacter pylori is statistically significant.

In some embodiments, there is provided an antimicrobial composition wherein the antimicrobial composition leads to a statistically significant clinical improvement in a human infected with a pathogenic urease active bacteria.

In some embodiments, there is provided an antimicrobial composition wherein the antimicrobial composition leads to a statistically significant clinical improvement in a human infected with Helicobacter pylori.

In an aspect of the present invention there is provided a use of an antimicrobial composition for the preparation or manufacture of a pharmaceutical formulation for separate, combined or concomitant administration designed for the treatment, alleviation or prophylaxis of GIT disease or disorder, gastritis, gastric ulcer, duodenal ulcer, gastric cancer, duodenal cancer in vertebrates, including mammals and birds, in need of such treatment, alleviation or prophylaxis.

In an aspect of the present invention there is provided a use of an antimicrobial composition for the preparation or manufacture of a pharmaceutical formulation for separate, combined or concomitant administration designed for the treatment, alleviation or prophylaxis of urinary tract infections (in particular those associated with Staphylococcus spp or Proteus spp infection), intestinal pneumonia, intestinal tuberculosis in vertebrates, including mammals and birds, in need of such treatment, alleviation or prophylaxis.

In an aspect of the present invention there is provided a use of an antimicrobial composition for the preparation or manufacture of a pharmaceutical formulation for separate, combined or concomitant administration designed for the treatment, alleviation or prophylaxis of liver disease, kidney disease in vertebrates, including mammals and birds, in need of such treatment, alleviation or prophylaxis. Both liver and kidney disease have been associated with high levels of ammonium in the gastrointestinal tract.

In some embodiments, the present invention is directed a use of an antimicrobial composition wherein the pharmaceutical formulation is in a form suitable for administering a dose of the antimicrobial composition in a dose or as divided doses or sub-doses administered at appropriate intervals per day.

In some embodiments, the present invention is directed a use of an antimicrobial composition wherein the pharmaceutical formulation in administered as one, two, three, four or more doses or sub-doses per day or per administration.

In some embodiments, the present invention is directed a use of an antimicrobial composition wherein the pharmaceutical formulation comprises from 107 to 109 CFU/g of Lactobacillus reuteri DSM17648 or Lactobacillus reuteri UBLRu-87 and zeolite, wherein the zeolite is present in an amount of at least 50 mg/gram of the formulation.

In some embodiments, the present invention is directed a use of an antimicrobial composition wherein the pharmaceutical formulation further comprises calcium carbonate and magnesium carbonate, wherein the calcium carbonate and magnesium carbonate are each present in an amount of at least 0.5 mg/gram of the formulation.

In some embodiments, the present invention is directed a use of an antimicrobial composition wherein the dose of the antimicrobial composition is formulated as a capsule, lyophilizate, liquid, pill, powder or gel.

In some embodiments, the present invention is directed a use of an antimicrobial composition wherein the treatment leads to complete remission of the disease or disorder caused by or associated with Helicobacter spp infection.

In some embodiments, the present invention is directed to an antimicrobial composition as described in the above paragraphs, for use in the reduction of ammonia in the gastrointestinal tract, preferably in the reduction of ammonia concentration in gastric juice.

The present inventors surprisingly and unexpectedly observed that the non-antibiotic antimicrobial composition of the present invention was effective at inactivating and treating Helicobacter pylori infection while established antibiotic therapies which rely on antibiotics, failed to do so. In particular, the present inventors surprisingly and unexpectedly observed that the non-antibiotic antimicrobial composition of the present invention was effective at inactivating and treating Helicobacter pylori infection by reducing predictive markers such as urea, gastric ammonia, acid reflux, bloating and urease activity, while antibiotic based therapies failed.

### BRIEF DESCRIPTION OF THE DRAWINGS

FIG. 1 In vitro urea and ammonia reduction assays using the antimicrobial compositions of the invention.

FIG. 2 Graph showing in vivo clinical test data.

FIG. 3 Graph showing Blank-adjusted absorbance of H. pylori Strain 83 and ATCC 700824 following 1:10 or 1:100 dilution in Brain Heart Infusion Broth +10 mM urea and 0.1% YE and incubation in microaerophilic conditions for 120 h.

FIG. 4 Graph showing total viable count of H. pylori Strain 83 and ATCC 700824 following 1:10 or 1:100 dilution in Brain Heart Infusion Broth +10 mM urea and 0.1% YE and incubation in microaerophilic conditions for 144 h.

FIG. 5 Graphs showing pH and ammonium concentration in Brain Heart Infusion broth+10% [v/v] FBS, 0.1% [w/v] yeast extract and 0, 10 or 50 mM urea initially adjusted to pH 2 (FIG. 5a), pH 5 (FIG. 5b) or unadjusted (pH 7) (FIG. 5c) following incubation in microaerophilic conditions for 8 h.

FIG. 6 Graphs showing pH and total viable count of H. pylori ATCC 700824 in Brain Heart Infusion broth +10% [v/v] FBS, 0.1% [w/v] yeast extract and 0, 10 or 50 mM urea initially adjusted to pH 2 (FIG. 6a), pH 5 (FIG. 6b) or unadjusted (pH 7) (FIG. 6c) following incubation in microaerophilic conditions for 8 h.

### DETAILED DESCRIPTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and the claims, the singular form “a,” “an” and “the” include singular and plural references unless the context clearly dictates otherwise. For example, the term “zeolite” includes a silicate containing substance with affinity for ammonia (NH4+). In the context of the present invention, a zeolite substance may be microporous or mesoporous silicate, known as a molecular sieve, which is granular or powdered for separate, combined or concomitant administration for use in the treatment, alleviation or prophylaxis of GIT disease or disorder, gastritis, gastric ulcer, duodenal ulcer, gastric cancer, duodenal cancer in vertebrates, including mammals and birds, in need of such treatment, alleviation or prophylaxis, alone or in a composition with at least one member of the Lactobacilli genus, once daily, or for several days, weeks, months or years, or more than once daily for days to years.

As used herein the term, “substance” refers to zeolite, pharmaceutical agents, therapeutically active ingredients and/or mineral salts such as for example calcium carbonate and/or magnesium carbonate and combinations thereof.

As used herein the term “antimicrobial” refers to destroying, killing or inhibiting the growth of microorganisms and especially pathogenic microorganisms such as pathogenic bacteria for example Helicobacter spp.

As used herein the abbreviation “(w/w)” means percent by weight, as calculated based on the weight of the component and the total weight of the composition or formulation. Generally, the terms used in this application are well known to persons of skill in the art.

All numbers or numerals as used herein that indicate for example amounts, ratios of materials or substances, physical properties of materials or substance, and/or use are to be understood as modified or qualified by the term “about,”except as otherwise explicitly indicated.

As used herein, the term “about” includes the recited number or number and +/−10% from the recited numeral or number. By way of non-limiting example, the term “about ten (10)” would encompass nine (9) to eleven (11) or 9-11.

As used herein, the term “subject” means any animal, such as a vertebrate, preferably a mammal such as human, to whom will be or has been administered substances, compounds or compositions according to embodiments of the invention. Preferably, a subject is in need of or has been the object of observation or experiment of, treatment or prevention of Helicobacter spp infection. Preferably, a subject is in need of or has been the object of observation or experiment of, treatment or prevention of Helicobacter spp infection such as Helicobacter canis, Helicobacter felis, Helicobacter heilmannii, Helicobacter mustelae, Helicobacter bizzozeronii, Helicobacter cinonyx, Helicobacter fenneliae, Helicobacter rappini, Helicobacter hepaticus, Helicobacter pullorum, Helicobacter bilis, Helicobacter rodentium, Helicobacter trogontum, Helicobacter cinaedi, Helicobacter muridarum, Helicobacter pametensis, Helicobacter cholecyctus and Helicobacter pylori infection. Preferably, a subject is in need of or has been the object of observation or experiment of, treatment or prevention of Helicobacter pylori infection.

As used herein the term “symptom” refers to for example vomiting, gastritis, peptic ulcers, gastric neoplasia, lymphoma, fever, GI disease, hormonal imbalance, associated with or caused by Helicobacter spp.

As used herein the term “condition” refers to for example hyperammonemia, associated with or caused by Helicobacter spp. It also relates to symptoms associated with infection by a urease active bacteria in the gastro-intestinal tract.

As used herein, the term “treatment” or “treating” refers to an amelioration, prophylaxis, or reversal of a disease or disorder, or of at least one discernible symptom or condition thereof. It is also contemplated that the treatment, as described herein throughout and based on data, leads to one or more of clinical improvement, reduction in the severity of a symptom or disease and reduction in one or more marker in a patient. Of one or more markers. In some embodiments, “treatment” or “treating” refers to an amelioration, prophylaxis, or reversal of at least one measurable physical parameter related to the disease or disorder being treated, not necessarily discernible in or by the mammal. In some embodiments, “treatment” or “treating” refers to inhibiting or slowing the progression of a disease, disorder or condition, either physically, e.g., stabilisation of a discernible symptom, physiologically, e.g., stabilisation of a physical parameter, or both. In some embodiments, “treatment” leads to partial or complete remission of the disease or disorder. In some embodiments, “treatment” leads to clinical improvement in a subject infected with or affected by Helicobacter spp such as Helicobacter pylori. In some embodiments, “treatment” leads to reduction in the severity of disease in a subject infected with or affected by Helicobacter spp such as Helicobacter pylori. In some embodiments, “treatment” leads to reduction in viral load in a subject infected with or affected by Helicobacter spp such as Helicobacter pylori. Without wishing to be bound by theory, elevated blood levels of different markers such as inflammatory markers, CBC markers and predictive markers have been associated with disease state or condition associated with Helicobacter spp such as Helicobacter pylori. Thus, in some embodiments of the present invention, the predictive markers are selected from the group consisting of the marker is a predictive marker selected from the group consisting of urea, gastric ammonia, acid reflux, bloating and urease activity. In some embodiments, the predictive marker is gastric ammonia. In some embodiments of the present invention, the treatment leads to reduction of one or more predictive markers selected from the group consisting of urea, gastric ammonia, acid reflux, bloating and urease activity. In some embodiments of the present invention, the treatment leads to reduction of the predictive marker gastric ammonia. In some embodiments of the present invention, the treatment leads to reduction or alleviation of hyperammonemia. H. pylori is strongly urease positive. Its ability to split urea within 30 seconds distinguishes it from other Helicobacter species. Bacteriology—Identification|ID 26|Issue no: 3|Issue date: 03.07.15|Page: 18 of 27. UK Standards for Microbiology Investigations |Issued by the Standards Unit, Public Health England. In some embodiments, “treatment” leads to clinical improvement in a subject infected with or affected by a urease-active bacteria, where the bacteria may be selected from Helicobacter spp, Staphylococcus spp., Proteus spp., Klebsiella spp., and Mycobacterium spp, more specifically by Helicobacter pylorus, Staphylococcus capitius urealiticum, Proteus mirabilis, Klebsiella pneumoniae, and/or Mycobacterium tuberculosis.

The term ‘urease-active bacteria’ refers to one or more bacteria that can convert urea to ammonia and carbon dioxide. This will result in increased urine pH. These organisms include Proteus, Nocardia, Ureaplasma, Helicobacter Pylori, Klebsiella, Cryptococcus, Staphylococcus Epidermidis, and Staphylococcus Saprophyticus. They may also be referred to a urease positive bacteria.

The present invention relates to a new composition and uses of the composition in treatment methods that alleviate, abrogate, or otherwise reduce or cure any one or more symptoms caused by or associated with a Helicobacter spp such as Helicobacter pylori infection. Specifically, the invention relates to a new composition and uses of the composition in treatment of Helicobacter spp such as Helicobacter pylori infected subjects. In some embodiments, the treatment leads to substantial cure of a disease or disorder caused by or associated with Helicobacter spp such as Helicobacter pylori infection. In some embodiments, the invention relates to a new composition and uses of the composition as palliation of Helicobacter spp such as Helicobacter pylori infected subjects.

While the emphasis of the present disclosure resides with subjects, those of skill in the art will readily recognise that the present invention is also equally applicable and effective to non-human subjects (i.e. vertebrate animals) such as, for example, livestock (e.g. cattle, horses, pigs and sheep), exotic animals (e.g. pandas, big cats such as tigers, lions and pumas, elephants, bats, mice, rats and similar animals) and also companion animals (such as dogs and cats), particularly where a disease or condition is associated with or caused by Helicobacter spp infection such as Helicobacter canis, Helicobacter felis, Helicobacter heilmannii, Helicobacter mustelae, Helicobacter bizzozeronii, Helicobacter cinonyx, Helicobacter fenneliae, Helicobacter rappini, Helicobacter hepaticus, Helicobacter pullorum, Helicobacter bilis, Helicobacter rodentium, Helicobacter trogontum, Helicobacter cinaedi, Helicobacter muridarum, Helicobacter pametensis, Helicobacter cholecyctus and Helicobacter pylori infection. In some embodiment of the present invention, the disease or condition is associated with or caused by Helicobacter canis.

The present inventors surprisingly and unexpectedly found that the non-antibiotic antimicrobial composition of the present invention was effective at inactivating and treating Helicobacter pylori infection while established antibiotic therapies which rely on antibiotics, failed to do so. In particular, the present inventors surprisingly and unexpectedly observed that the non-antibiotic antimicrobial composition of the present invention was effective at inactivating and treating Helicobacter pylori infection by reducing predictive markers such as urea, gastric ammonia, acid reflux, bloating and urease activity, while antibiotic based therapies failed for example due to antibiotic resistance.

As used herein, the term “non-antibiotic” refers to not using, relying on or containing an antibiotic. In some embodiments, the composition of the present invention does not use, rely on or contain an antibiotic.

As used herein the term “antibiotic” refers to an antibacterial substance (such as penicillin, cephalosporin, metronidazole, clarithromycin, furazolidone, amoxicillin, tetracycline, ciprofloxacin, levofloxacin) that is used to treat or prevent infections by killing or inhibiting the growth of bacteria in or on the subject, that is administered orally, topically, or by injection, and that is isolated from cultures of certain microorganisms (such as fungi) or is of semi-synthetic or synthetic origin. Currently, a standard triple therapy consisting of two antibiotics and a proton-pump inhibitor have been proposed as the first-line regimen. Alternatively, bismuth-containing quadruple treatment, sequential treatment or a non-bismuth quadruple treatment (concomitant) are also used in current treatment therapy. Levofloxacin containing triple treatment are recommended as rescue treatment for infection of H. pylori after defeat of first-line therapy. However, due to a rapid acquisition of antibiotic resistance drastically reducing the effectiveness of any antibiotic regimens inevitably leading to acute and long-term suffering with concomitant and often unpredictable side effects for the affected subject. It is known that H. pylori is becoming increasingly resistant to metronidazole and clarithromycin. Bacteriology—Identification|ID 26|Issue no: 3|Issue date: 03.07.15|Page: 10 of 27. UK Standards for Microbiology Investigations|Issued by the Standards Unit, Public Health England.

As contemplated in the context of the present invention, an antibiotic can be used to treat or prevent Helicobacter spp infection such as Helicobacter pylori infection. In some embodiments, the Helicobacter spp, such as Helicobacter canis, Helicobacter felis, Helicobacter heilmannii, Helicobacter mustelae, Helicobacter bizzozeronii, Helicobacter cinonyx, Helicobacter fenneliae, Helicobacter rappini, Helicobacter hepaticus, Helicobacter pullorum, Helicobacter bilis, Helicobacter rodentium, Helicobacter trogontum, Helicobacter cinaedi, Helicobacter muridarum, Helicobacter pametensis, Helicobacter cholecyctus and Helicobacter pylori is resistant to antibiotic treatment. In some embodiments Helicobacter pylori is resistant to antibiotic treatment.

As used herein the term “administration” should be understood to encompass for example peroral, intravenous, parenteral, inhalation, pulmonary, rectal, nasal, topical, intravesical, intrathecal, enteral, intralymphatic, intracavital, vaginal, transurethral, intradermal, aural, intramammary, buccal, orthotopic, intratracheal, intralesional, percutaneous, endoscopical, transmucosal, sublingual, intestinal administration and combinations thereof. In some embodiments, the regimen of administration of the antimicrobial composition is suitable for peroral, rectal, topical, enteral, vaginal, buccal, orthotopic, intratracheal, intralesional, endoscopical, transmucosal, sublingual, intestinal administration and combinations thereof. In some embodiments, the regimen of administration of the antimicrobial composition or formulation is peroral.

In some embodiment, the zeolite is a silicate containing substance with affinity for ammonia (NH4+). In some embodiments, the zeolite is synthetic zeolite with affinity for ammonia (NH4+). As used herein, “synthetic zeolite” refers to a zeolite manufactured or synthesized by one or more chemical reactions involving breaking and/or making chemical bonds. Physicochemical properties and features of zeolites include for example different size pores, channels and ion exchange capacity. The channels are filled with water and with exchangeable cations to balance negative charges in the zeolite structural framework. As a consequence of the highly porous structure and the substitution, clinoptilolite and other zeolites have significant cation exchange capacities (CEC), on the order of 100 to 300 cmol of charge per kg. As with most zeolites, however, exchange selectivity is restricted by channel size. Larger ions may be selectively excluded. These “ion sieving” properties of zeolites are important properties exploited in a number of industrial and other uses. In the presence of ammonium rich liquids, NH4+-N is exchanged with the cations present in the framework.

In some embodiments, there is provided an antimicrobial composition wherein the zeolite is zeolite clinoptilolite (ZC). In some embodiments, there is provided an antimicrobial composition wherein the zeolite clinoptilolite comprises Clinoptilolite-K, Clinoptilolite-Na and Clinoptilolite-Ca. In some embodiments, there is provided an antimicrobial composition wherein the zeolite is activated zeolite clinoptilolite (aZC). In some embodiment, there is provided an antimicrobial composition wherein activated zeolite clinoptilolite (aZC) is double activated zeolite clinoptilolite (aZC). The clinoptilolite series comprises three known species: Clinoptilolite-K, Clinoptilolite-Na, Clinoptilolite-Ca named after their dominant chemical element constituent namely K+, Na+and Ca++. These chemical element constituents are exchanged during cation exchange in favour of for example heavy metals, toxins, mycotoxins and ammonia as present in the environment, such as the GIT, displaying higher chemical affinity for zeolite clinoptilolite such as Clinoptilolite-K, Clinoptilolite-Na and Clinoptilolite-Ca.

In some embodiment the Activated Zeolite Clinoptilolite is Tribomechanically Activated Zeolite Clinoptilolite.

In some embodiment the Tribomechanically Activated Zeolite Clinoptilolite is (TMAZ®). It would be known by the skilled person that the chemical formula of SiO2, 65.0-71.3%; Al2O3, 11.5-13.1%; CaO 2.7-5.2%; K2O, 2.2-3.4%; Fe2O3, 0.7-1.9%; MgO, 0.6-1.2%; Na2O, 0.2-1.3%; TiO2, 0.1-0.3%; Si/Al ratio, 4.8-5.4.

It would be understood by the skilled person that the empirical formula of zeolite can be (Ca, K2, Na2, Mg)4Al8Si40O96×24H2O with specific molecular mass, 2.2-2.5 g/cm3; porosity 32- 40%; effective pore diameter, 0.4 nm or greater such as 0.5 nm, 0.75 nm, 1 nm, 1.5 nm, 2 nm, 10 nm, 25 nm, 50 nm 100 nm, 150 nm, 250 nm, 0.5 m or grater diameter.

In some embodiments the zeolite displays Ion-exchange capacity: 1.2-1.5 mol/kg; Ca2+, 0.64-0.98 mol/kg; Mg2+, 0.06-0.19 mol/kg; K+, 0.22-0.45 mol/kg; Na+, 0.01-0.19 mol/kg Ion-exchanging selectivity Cs>NH4+>Pb2+>K+>Na+>Mg2+>Ba2+>Cu2+>Zn2+.

In some embodiments, the chemicals or toxins absorbed by zeolite comprise NH3, hydrocarbons C1-C4, CO2, H2S, SO2, NOX, Aldehydes.

Preferably, as contemplated within the context of the compositions or formulations of the present invention, zeolite is nontoxic. In some embodiment zeolite is generally recognized as safe (GRAS) according to US Code of Federal Regulations (21 CFR 182, Subpart C).

It is proposed that the antimicrobial composition and uses of the composition in treatment methods are able to alleviate, abrogate, or otherwise reduce or cure any one or more symptoms caused by or associated with a Helicobacter spp such as Helicobacter pylori infection by restoring the natural acid pH levels in the GIT of the subject.

It is proposed that the mineral salts in the composition of the present invention such as for example calcium carbonate and magnesium carbonate, further extend or contribute to the ion exchange capability of the zeolite, thereby affording an enhancement in the absorption and/or neutralisation of ammonium such as ammonium in the GIT.

As used herein, “palliation” refers to any form of medical care or treatment that concentrates on reducing the severity of the symptoms of a disease or slows its progress rather than providing a cure. It aims at improving quality of life, and particularly at reducing or eliminating pain. The definition specifically focuses on the general unavailability of a cure in that it emphasizes the active total care of subject whose disease is not responsive to curative treatment.

Without wishing to be bound by theory, a disease or condition associated with or caused by Helicobacter spp infection such as Helicobacter canis, Helicobacter felis, Helicobacter heilmannii, Helicobacter mustelae, Helicobacter bizzozeronii, Helicobacter cinonyx, Helicobacter fenneliae, Helicobacter rappini, Helicobacter hepaticus, Helicobacter pullorum, Helicobacter bilis, Helicobacter rodentium, Helicobacter trogontum, Helicobacter cinaedi, Helicobacter muridarum, Helicobacter pametensis, Helicobacter cholecyctus and Helicobacter pylori infection is characterised with increased levels or higher than normal levels of urea, gastric ammonia, acid reflux, bloating and urease activity. Gastric Helicobacter spp infection has been reported to be associated with hyerammonemia. H. pylori, H. bizzozeroni, H. cynogastricus, H. salomonis, H. suis, H. bilis, H. heilmannii are urease positive. Bacteriology—Identification|ID 26|Issue no: 3|Issue date: 03.07.15|Page: 21 of 27. UK Standards for Microbiology Investigations|Issued by the Standards Unit, Public Health England.

As used herein the term “hyerammonemia” or other terms used to indicate higher than normal levels of ammonia is meant higher levels than normal, where normal is a specified laboratory's normal range as determined for blood from non-diseased, healthy subjects, using an analytical technique-of-choice for that laboratory that performed the assay (which could be venous blood, arterial blood, or some fraction of whole blood including plasma or serum) and processed according to the laboratory's procedure specifying whether it should be placed immediately on ice, the timeframe in which it is to be delivered to the laboratory, using the specified preservative, if any.

Lactic acid bacteria, especially Lactobacillus, are the most commonly used microorganisms as probiotics—“Generally Recognized As Safe” (GRAS). Acidity, presence of ammonia, bile salts, and pancreatic enzymes in the gastrointestinal tract (GIT) are some of the major stresses that an orally taken probiotic experiences in the GIT. Apart from being able to survive, a probiotic strain of the present invention also has to be able to adhere to and subsequently colonize (at least temporarily) the intestinal tract. Since the GIT is a dynamic environment, the flow of the gut digesta may wash out any bacterium not attached to the intestinal mucosa. Thus, probiotic strains with adherent ability are more likely to have an increased opportunity to colonize the GIT and provide beneficial effects. In some embodiments, the Lactobacillus is able to withstand, preferably remain active or biochemically effective, at elevated levels of gastric ammonia observed with Helicobacter spp infection such as Helicobacter pylori infection.

Different methods of producing probiotics have been described and are well documented. Some of these methods describe producing mixed microbial cultures in liquid growth medium. The production of such mixed cultures is greatly challenged by microbial competition among the diverse populations in the culture medium. One way of producing mixed microbial cultures is to separately propagate the different strains and to afterwards combine the propagated strains in the desired ratio.

Other methods of propagating probiotics focus on the quorum sensing capabilities of microorganisms, particularly bacteria such as lactic acid bacteria. Lactic acid bacteria, due to the metabolic stress they experience, increase the release of different antimicrobial metabolites. In particular, a combination of metabolic stress and quorum sensing capabilities of the microorganisms, maximise the release of different antimicrobial metabolites (WO2020/177858).

In the context of the present invention, the at least one member of the Lactobacilli genus is produced using methods known in the art. In some embodiments, the at least one member of the Lactobacilli genus is produced using methods known in the art.

In some embodiments, the at least one member of the Lactobacilli genus is propagated or produced using a combination of metabolic stress and quorum sensing capabilities of the at least one member of the Lactobacilli genus.

In some embodiments, the at least one member of the Lactobacilli genus is selected from the group consisting of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii. In some embodiment, the at least one member of the Lactobacilli genus is selected from the group consisting of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii is propagated or produced using a combination of metabolic stress and quorum sensing capabilities of the at least one member of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii. In some embodiment, the at least one member of the Lactobacilli genus is selected from the group consisting of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii.

In some embodiments, the at least one member of the Lactobacilli genus is Lactobacillus reuteri. In some embodiments, the at least one member of Lactobacillus reuteri is Lactobacillus reuteri DSM17648. In some embodiments, Lactobacillus reuteri DSM17648 is produced or propagated using a combination of metabolic stress and the quorum sensing capabilities of Lactobacillus reuteri DSM17648.

Furthermore, in WO 2007/073709 a number of other suitable Lactobacillus strains are identified which are described in the German Collection of Microorganisms and Cell Cultures GmbH (DSMZ), Mascheroder Weg 1b, D-38124 Braunschweig, Germany, namely: DSM 17646, DSM 17647, DSM 17649, DSM 17650, DSM 17651, DSM 17652 and DSM 17653. In some embodiments the at least one member of the Lactobacilli genus is Lactobacillus reuteri UBLRu-87 (MTCC 5403).

The antimicrobial metabolites can be toxic to pathogenic microorganisms such as bacterial pathogens. In some embodiments, the composition or formulation of the present invention comprises antimicrobial metabolites which are toxic to Helicobacter spp such as Helicobacter pylori.

In some embodiments, the at least one member of the Lactobacilli genus of the composition of the present invention is lyophilised. In some embodiments, the at least one member of the Lactobacilli genus of the composition of the present invention is freeze dried. In some embodiments the at least one member of the Lactobacilli genus of the composition of the present invention is viable.

As used in the present invention, a therapeutically effective amount of the composition comprising of the present invention can be delivered to a patient as part of a regimen.

In some embodiments, there is provided an antimicrobial composition wherein the regimen comprises one or more types of administration of the zeolite and the at least one member of the Lactobacilli genus. In some embodiments, the regimen of administration of the antimicrobial composition is suitable for peroral, rectal, topical, enteral, vaginal, buccal, orthotopic, intratracheal, intralesional, endoscopical, transmucosal, sublingual, intestinal administration and combinations thereof. In some embodiments, the antimicrobial composition is suitable for peroral administration.

As used herein the term “regimen” refers to a plan or a set of rules of different possible routs or modes of administration, preferably to achieve a therapeutically effective amount of the antimicrobial composition of the present invention. It would be known to those of skill in the art that different regimens may be employed in the context of the present invention.

In some embodiments, the regimen of administration of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is carried out using methods known in the art.

As used herein the term “administration” should be understood to encompass for example peroral, rectal, topical, enteral, vaginal, buccal, orthotopic, intratracheal, intralesional, endoscopical, transmucosal, sublingual, intestinal administration and combinations thereof.

Peroral Administration: The composition of the invention can be formulated to take the form of tablets or capsules prepared by conventional means with one or more pharmaceutically acceptable carriers (e.g., excipients such as binding agents, fillers, lubricants and disintegrants). The inventors found that the composition may be conveniently administered to a subject by the peroral route, particularly in the form of a tablet or capsule (e.g. a tablet). In some embodiments, the particular dosage regimes contemplated in the invention are particularly suited to oral administration in the form of a tablet or capsule that is formulated such that the release of compounds used in the invention e.g. zeolite, and the at least one member of the Lactobacilli genus, calcium carbonate and magnesium carbonate. The term “modified” or “modified release” as used herein in relation to the composition according to the invention or as used in any other context means release, which is not immediate release and is taken to encompass controlled release, sustained release, prolonged release, timed release, retarded release, extended release and delayed release.

Controlled-Release Administration: Controlled-release (or sustained-release) preparations can be formulated to extend the activity of a substance and reduce dosage frequency. Controlled-release preparations can also be used to affect the time of onset of action or other characteristics, such as blood levels of the substance, and consequently affect the occurrence of any side effects.

Controlled-release preparations can be designed to initially release an amount of a substance that produces the desired therapeutic effect, and gradually and continually release other amounts of the substance to maintain the level of therapeutic effect over an extended period of time. In order to maintain a near-constant level of a substance in the body, the substance can be released from the dosage form at a rate that will replace the amount of the composition of the present invention being metabolised and/or excreted from the body. The controlled-release can be stimulated by various inducers, e.g., change in pH, change in temperature, enzymes, water, and/or other physiological conditions or molecules such as ammonia.

Controlled-release systems can include, for example, an infusion pump which can be used to administer the composition in a manner similar to that used for delivering insulin or chemotherapy to the body generally, or to specific organs or tumours. Typically, using such a system, the composition is administered in combination with a biodegradable, biocompatible polymeric implant that releases the composition over a controlled period of time at a selected site. Example polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target such as a GIT ulcer, thus requiring only a fraction of a peroral dosage.

Compositions of the invention can be administered by other controlled-release means or delivery devices that are well known to those of skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of the composition or formulation of the invention will be known to the skilled person and are within the scope of the invention. It will be appreciated by those skilled in the art that the amount of the different constituents of the composition of the invention required in the present uses and methods will vary with the nature or severity of the condition being treated, the age, weight, pre-existing medical treatments, and the overall condition of the subject.

In some embodiments, there is provided an antimicrobial composition wherein the antimicrobial composition further comprises biologically active substances or minerals such as calcium carbonate, magnesium carbonate, vitamins, such as D and E, pharmaceutically acceptable carriers, additives and adjuvants, as well as antimicrobial chitin, such as chitosan and alpha-ketoglutarate, citrate and lactate.

In some embodiments, there is provided an antimicrobial composition wherein the regimen of administration of the zeolite and the at least one member of the Lactobacilli genus, is suitable for peroral administration or gastric administration.

In some embodiments, there is provided an antimicrobial composition wherein the composition comprises, activated zeolite clinoptilolite (aZC), Lactobacillus reuteri DSM17648 or Lactobacillus reuteri UBLRu-87, calcium carbonate and magnesium carbonate.

In some embodiments, there is provided an antimicrobial composition wherein the antimicrobial composition leads to a reduction of one or more markers.

In some embodiments, there is provided an antimicrobial composition wherein the marker is a predictive marker selected from the group consisting of urea, gastric ammonia, acid reflux, bloating and urease activity.

In some embodiments, there is provided an antimicrobial composition wherein the predictive marker is gastric ammonia.

In some embodiments, there is provided an antimicrobial composition wherein the antimicrobial composition leads to reduction of gastric ammonia levels in a vertebrate infected by Helicobacter pylori.

In some embodiments, there is provided an antimicrobial composition wherein the antimicrobial composition leads to reduction of gastric ammonia levels in a vertebrate infected by Staphylococcus spp. e.g. Staphylococcus capitius urealiticum infection; Proteus spp. e.g. Proteus mirabilis infection; Klebsiella spp. e.g. Klebsiella pneumoniae infection, and Mycobacterium spp. e.g. Mycobacterium tuberculosis infection.

In some embodiments, there is provided an antimicrobial composition wherein the reduction of gastric ammonia is at least 5%, 10%, 15%, 20%, 25%, 30%, 35%, 40%, 45%, 50%, 55%, 60%, 65%, 70%, 75%, 80%, 85%, 90%, 95% or 100% or greater compared to placebo.

In some embodiments, there is provided an antimicrobial composition wherein the reduction of gastric ammonia is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to placebo.

In some embodiments, there is provided an antimicrobial composition wherein the reduction of the predictive marker is statistically significant compared to a placebo.

In some embodiments, there is provided an antimicrobial composition wherein the reduction of gastric ammonia marker levels in a human infected with Helicobacter pylori is statistically significant. In some embodiments, there is provided an antimicrobial composition wherein the antimicrobial composition leads to a statistically significant clinical improvement in a human infected with Helicobacter pylori.

The term “regression” may be used interchangeably with the terms, “reduction”, “decrease” or “reduce” a disease or disorder, viral load, or of at least one discernible symptom or marker caused by or associated with Helicobacter spp such as Helicobacter pylori infection. In some embodiments, the reduction is at least 5%, 10%, 15%, 20%, 25%, 30%, 35%, 40%, 45%, 50%, 55%, 60%, 65%, 70%, 75%, 80%, 85%, 90%, 95% or 100% or greater compared to control or placebo. In some embodiments the reduction is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or placebo. In some embodiments the regression or reduction is statistically significant. In some embodiments the effectiveness or the treatment of the present invention is assessed by a quantifying step. In some embodiments, regression is assessed by a quantifying step. In further embodiments, the quantifying step is performed on a sample. In further embodiments, the quantifying step is performed by an immunoassay. In some embodiments, the sample is one of a plasma sample, blood sample, sputum sample, lavage, synovial fluid, or combinations thereof.

In some embodiments, regression refers to a disease or disorder, or at least one discernible symptom caused by or associated with Helicobacter spp. such as Helicobacter pylori infection. In some embodiments, regression refers to a disease or disorder, or at least one discernible symptom caused by or associated with a urease active bacterial infection, including Staphylococcus spp. e.g. Staphylococcus capitius urealiticum infection; Proteus spp. e.g. Proteus mirabilis infection; Klebsiella spp. e.g. Klebsiella pneumoniae infection, and Mycobacterium spp. e.g. Mycobacterium tuberculosis infection. In some embodiments, regression is used to provide an indication of the extent by which, for example, disease, disorder or symptom is altered in terms of, for example, frequency of occurrence, intensity and severity. In some embodiments, regression means make less, bring down, lower or lessen, for example, fever in the treated subject. In some embodiments, reduction means make less, bring down, lower or lessen, the viral load in the treated patient. In some embodiments, the present treatment leads to an accelerated or faster reduction in the viral load in the treated patient compared to a placebo. In some embodiments, the reduction of the viral load is statistically significant.

As used herein, the term “significant” when referring to for example reducing, enhancing, remission, amelioration, prophylaxis, or reversal, that is statistically significant, not due to chance alone, which has a p-value of 0.05 or less. In particular, the term “significant” can have a p-value of less than 0.05, 0.04, 0.03, 0.01, 0.005, 0.001, etc., when referring to for example reducing, enhancing, remission, amelioration, prophylaxis, or reversal of disease, disorder or symptom caused by or associated with Helicobacter spp such as Helicobacter pylori infection, for example when compared with the level or frequency of occurrence of implantation or pregnancy in one or more non-treated patients or when compared to the level or frequency of occurrence of implantation or pregnancy in the same patient observed at an earlier time point (e.g. comparison with a “base line” level or placebo). Those of skill in the relevant art would be familiar with different statistical calculation approaches, examples include, t-test, z-test, sample test, O'Brien-Fleming method for normally distributed data etc.

As used herein, the term “significant” when referring to for example reducing, enhancing, remission, amelioration, prophylaxis, or reversal, that is statistically significant, not due to chance alone, which has a p-value of 0.05 or less. In particular, the term “significant” can have a p-value of less than 0.05, 0.04, 0.03, 0.01, 0.005, 0.001, etc., when referring to for example reducing, enhancing, remission, amelioration, prophylaxis, or reversal of disease, disorder or symptom caused by or associated with Staphylococcus spp. e.g. Staphylococcus capitius urealiticum infection; Proteus spp. e.g. Proteus mirabilis infection; Klebsiella spp. e.g. Klebsiella pneumoniae infection, and Mycobacterium spp. e.g. Mycobacterium tuberculosis infection, for example when compared with the level or frequency of occurrence of implantation or pregnancy in one or more non-treated patients or when compared to the level or frequency of occurrence of implantation or pregnancy in the same patient observed at an earlier time point (e.g. comparison with a “base line” level or placebo). Those of skill in the relevant art would be familiar with different statistical calculation approaches, examples include, t-test, z-test, sample test, O'Brien-Fleming method for normally distributed data etc.

In some embodiments the significant reducing, enhancing, remission, amelioration, prophylaxis, or reversal, refers to statistically significant reduction, enhancement, remission, amelioration, prophylaxis, or reversal of disease, disorder or symptom caused by or associated with Helicobacter spp such as Helicobacter pylori infection.

In an aspect of the present invention there is provided a use of an antimicrobial composition for the preparation or manufacture of a pharmaceutical formulation for separate, combined or concomitant administration designed for the treatment, alleviation or prophylaxis of GIT disease or disorder, gastritis, gastric ulcer, duodenal ulcer, gastric cancer, duodenal cancer in vertebrates, including mammals and birds, in need of such treatment, alleviation or prophylaxis.

Without wishing to be bound by theory, the inventors have hypothesised that the body naturally creates more gastric acid to combat H. pylori in an inflammatory response. The body also recognises when this acid becomes too high it needs buffered so uses stores of calcium and magnesium to achieve this, over time too much acid and depleted stores of magnesium and calcium create many other ailments alongside the gastric disorders created by the auto immune response and colonisation of the H. pylori. Using the non-antibiotic antibacterial compositions of the present invention were not only able to effectively show experimentally reduction of ammonia and urea levels in affected subjects such as human, but also show complete elimination of the pathogen H. pylori thereby alleviating and/or preventing any H. pylori linked symptoms, diseases and conditions.

It is further hypothesises that the same situation applies to other urease positive bacterial infections in the gastrointestinal tract.

In some embodiments, the present invention is directed a use of an antimicrobial composition wherein the pharmaceutical formulation is in a form suitable for administering a dose of the antimicrobial composition in a dose or as divided doses or sub-doses administered at appropriate intervals per day.

In some embodiments, the present invention is directed a use of an antimicrobial composition wherein the pharmaceutical formulation in administered as one, two, three, four or more doses or sub-doses per day or per administration.

In some embodiments, the present invention is directed a use of an antimicrobial composition wherein the pharmaceutical formulation comprises from 107 to 109 CFU/g of Lactobacillus reuteri DSM17648 or Lactobacillus reuteri UBLRu-87, and zeolite, wherein the zeolite is present in an amount of at least 50 mg/gram of the formulation.

In some embodiments, the zeolite will typically be in the range of about 0.1 to 2000 mg/kg of body weight, about 0.15 to 1750 mg/kg of body weight, about 0.2 to 1700 mg/kg of body weight, about 0.3 to 1500 mg/kg of body weight, about 0.5 to 1250 mg/kg of body weight, about 1 to 1000 mg/kg of body weight, about 2 to 900 mg/kg of body weight, about 3 to 800 mg/kg of body weight, about 4 to 700 mg/kg of body weight, about 5 to 600 mg/kg of body weight, about 10 to 500 mg/kg of body weight, administered as one, two, three, four or more doses or sub-doses per day or per administration. In some embodiments, the dose is 400 mg/kg of body weight administered as one, two, three, four or more doses or sub-doses per day or per administration.

In some embodiments, the present invention is directed a use of an antimicrobial composition wherein the pharmaceutical formulation further comprises calcium carbonate and magnesium carbonate, wherein the calcium carbonate and magnesium carbonate are each present in an amount of at least 0.5 mg/gram of the composition or the formulation.

In some embodiments, the calcium carbonate and magnesium carbonate will typically be in the range of about 0.1 to 200 mg/kg of body weight, about 0.15 to 150 mg/kg of body weight, about 0.2 to 120 mg/kg of body weight, about 0.3 to 100 mg/kg of body weight, about 0.5 to 75 mg/kg of body weight, about 1 to 50 mg/kg of body weight, about 2 to 40 mg/kg of body weight, about 3 to 30 mg/kg of body weight, about 4 to 20 mg/kg of body weight, about 5 to 10 mg/kg of body weight, administered as one, two, three, four or more doses or sub-doses per day or per administration.

In some embodiments, the present invention is directed a use of an antimicrobial composition wherein the dose of the antimicrobial composition is formulated as a capsule, lyophilizate, liquid, pill, powder or gel.

In some embodiments, the present invention is directed a use of an antimicrobial composition wherein the treatment leads to complete remission of the disease or disorder caused by or associated with Helicobacter spp infection.

In some embodiments, the composition contains about 1010 cells/ml of the at least one member of the Lactobacilli genus. In some embodiments, the inoculated aqueous medium contains about 109 cells/ml of the at least one member of the Lactobacilli genus. In some embodiments, the inoculated aqueous medium contains about cells/ml of the at least one member of the Lactobacilli genus. In some embodiments, the inoculated aqueous medium contains about 107 cells/ml of the at least one member of the Lactobacilli genus. In some embodiments, the inoculated aqueous medium contains about cells/ml of the at least one member of the Lactobacilli genus. In some embodiments, the inoculated aqueous medium contains about 105 cells/ml of the at least one member of the Lactobacilli genus.

In some embodiments, there is provided an antimicrobial wherein the at least one member of the Lactobacilli genus is viable.

The skilled person would know that viability of microbial cells can be assessed or estimated using colony-forming units per millilitre (CFU/mL) in case of a liquid being tested or grams (CFU/g) if a solid material is tested.

In some embodiments, where the at least one member of the Lactobacilli genus composition is a liquid, the concentration of the viable cells can be estimated via colony-forming units per millilitre (CFU/mL or growth medium). In some embodiments, the concentration of the viable cells in the composition is from 0.5 million to 1 billion CFU/mL, 0.5 million to 500 million CFU/mL, 0.5 million to 400 million CFU/mL, 0.5 million to 300 million CFU/mL, 0.5 million to 200 million CFU/mL, 0.5 million to 150 million CFU/mL, 0.5 million to 125 million CFU/mL, 0.5 million to 100 million CFU/mL, 0.5 million to 75 million CFU/mL, 0.5 million to 50 million CFU/mL, 0.5 million to 10 million CFU/mL, 0.5 million to 5 million CFU/mL, 0.5 million to 1 million CFU/mL, 1 million to 1 billion CFU/mL, 1 million to 500 million CFU/mL, 1 million to 400 million CFU/mL, 1 million to 300 million CFU/mL, 1 million to 200 million CFU/mL, 1 million to 150 million CFU/mL, 1 million to 125 million CFU/mL, 1 million to 100 million CFU/mL, 1 million to 75 million CFU/mL, 1 million to 50 million CFU/mL, 1 million to 10 million CFU/mL, 1 million to 5 million CFU/mL, 5 million to 1 billion CFU/mL, 5 million to 500 million CFU/mL, 5 million to 400 million CFU/mL, 5 million to 300 million CFU/mL, 5 million to 200 million CFU/mL, 5 million to 150 million CFU/mL, 5 million to 125 million CFU/mL, 5 million to 100 million CFU/mL, 5 million to 75 million CFU/mL, 5 million to 50 million CFU/mL, 5 million to 10 million CFU/mL, 10 million to 1 billion CFU/mL, 10 million to 500 million CFU/mL, 10 million to 400 million CFU/mL, 10 million to 300 million CFU/mL, 10 million to 200 million CFU/mL, 10 million to 150 million CFU/mL, 10 million to 125 million CFU/mL, 10 million to 100 million CFU/mL, 10 million to 75 million CFU/mL, 10 million to 50 million CFU/mL, 50 million to 1 billion CFU/mL, 50 million to 500 million CFU/mL, 50 million to 400 million CFU/mL, 50 million to 300 million CFU/mL, 50 million to 200 million CFU/mL, 50 million to 150 million CFU/mL, 50 million to 125 million CFU/mL, 50 million to 100 million CFU/mL, 50 million to 75 million CFU/mL, 100 million to 1 billion CFU/mL, 100 million to 500 million CFU/mL, 100 million to 400 million CFU/mL, 100 million to 300 million CFU/mL, 100 million to 200 million CFU/mL, 100 million to 150 million CFU/mL, 100 million to 125 million CFU/mL, 125 million to 1 billion CFU/mL, 125 million to 500 million CFU/mL, 125 million to 400 million CFU/mL, 125 million to 300 million CFU/mL, 125 million to 200 million CFU/mL, 125 million to 150 million CFU/mL, 150 million to 1 billion CFU/mL, 150 million to 500 million CFU/mL, 150 million to 400 million CFU/mL, 150 million to 300 million CFU/mL, 150 million to 200 million CFU/mL, 200 million to 1 billion CFU/mL, 200 million to 500 million CFU/mL, 200 million to 400 million CFU/mL, 200 million to 300 million CFU/mL, 300 million to 1 billion CFU/mL, 300 million to 500 million CFU/mL, 300 million to 400 million CFU/mL, 400 million to 1 billion CFU/mL, 400 million to 500 million CFU/mL, or 500 million to 1 billion CFU/mL or more such as 10 billion CFU/ml.

In some embodiments, where the composition is a solid, the concentration of the viable at least one member of the Lactobacilli genus cells can be estimated via colony-forming units per gram (CFU/g). In some embodiments, the concentration of the at least one member of the Lactobacilli genus is from 0.5 million to 1 billion CFU/g, 0.5 million to 500 million CFU/g, 0.5 million to 400 million CFU/g, 0.5 million to 300 million CFU/g, 0.5 million to 200 million CFU/g, 0.5 million to 150 million CFU/g, 0.5 million to 125 million CFU/g, 0.5 million to 100 million CFU/g, 0.5 million to 75 million CFU/g, 0.5 million to 50 million CFU/g, 0.5 million to 10 million CFU/g, 0.5 million to 5 million CFU/g, 0.5 million to 1 million CFU/g, 1 million to 1 billion CFU/g, 1 million to 500 million CFU/g, 1 million to 400 million CFU/g, 1 million to 300 million CFU/g, 1 million to 200 million CFU/g, 1 million to 150 million CFU/g, 1 million to 125 million CFU/g, 1 million to 100 million CFU/g, 1 million to 75 million CFU/g, 1 million to 50 million CFU/g, 1 million to 10 million CFU/g, 1 million to 5 million CFU/g, 5 million to 1 billion CFU/g, 5 million to 500 million CFU/g, 5 million to 400 million CFU/g, 5 million to 300 million CFU/g, 5 million to 200 million CFU/g, 5 million to 150 million CFU/g, 5 million to 125 million CFU/g, 5 million to 100 million CFU/g, 5 million to 75 million CFU/g, 5 million to 50 million CFU/g, 5 million to 10 million CFU/g, 10 million to 1 billion CFU/g, 10 million to 500 million CFU/g, 10 million to 400 million CFU/g, 10 million to 300 million CFU/g, 10 million to 200 million CFU/g, 10 million to 150 million CFU/g, 10 million to 125 million CFU/g, 10 million to 100 million CFU/g, 10 million to 75 million CFU/g, 10 million to 50 million CFU/g, 50 million to 1 billion CFU/g, 50 million to 500 million CFU/g, 50 million to 400 million CFU/g, 50 million to 300 million CFU/g, 50 million to 200 million CFU/g, 50 million to 150 million CFU/g, 50 million to 125 million CFU/g, 50 million to 100 million CFU/g, 50 million to 75 million CFU/g, 100 million to 1 billion CFU/g, 100 million to 500 million CFU/g, 100 million to 400 million CFU/g, 100 million to 300 million CFU/g, 100 million to 200 million CFU/g, 100 million to 150 million CFU/g, 100 million to 125 million CFU/g, 125 million to 1 billion CFU/g, 125 million to 500 million CFU/g, 125 million to 400 million CFU/g, 125 million to 300 million CFU/g, 125 million to 200 million CFU/g, 125 million to 150 million CFU/g, 150 million to 1 billion CFU/g, 150 million to 500 million CFU/g, 150 million to 400 million CFU/g, 150 million to 300 million CFU/g, 150 million to 200 million CFU/g, 200 million to 1 billion CFU/g, 200 million to 500 million CFU/g, 200 million to 400 million CFU/g, 200 million to 300 million CFU/g, 300 million to 1 billion CFU/g, 300 million to 500 million CFU/g, 300 million to 400 million CFU/g, 400 million to 1 billion CFU/g, 400 million to 500 million CFU/g, or 500 million to 1 billion CFU/g, or more such as 10 billion CFU/ml.

In some embodiments, the present invention is directed a use of an antimicrobial composition wherein the pharmaceutical formulation comprises from 107 to 109 CFU/g of Lactobacillus reuteri DSM17648 or Lactobacillus reuteri UBLRu-87 and zeolite, wherein the zeolite is present in an amount of at least 50 mg/gram of the formulation.

In some embodiments, the present invention is directed a use of an antimicrobial composition wherein the pharmaceutical formulation further comprises calcium carbonate and magnesium carbonate, wherein the calcium carbonate and magnesium carbonate are each present in an amount of at least 0.5 mg/gram of the formulation.

In some embodiments, the present invention is directed a use of an antimicrobial composition wherein the dose of the antimicrobial composition is formulated as a capsule, lyophilizate, liquid, pill, powder or gel.

In some embodiments, the present invention is directed a use of an antimicrobial composition wherein the treatment leads to complete remission of the disease or disorder caused by or associated with Helicobacter spp infection.

In some embodiments, the present invention is directed a use of an antimicrobial composition wherein the treatment leads to complete remission of the disease or disorder caused by or associated with a Staphylococcus spp. e.g. Staphylococcus capitius urealiticum infection; Proteus spp. e.g. Proteus mirabilis infection; Klebsiella spp. e.g. Klebsiella pneumoniae infection, and Mycobacterium spp. e.g. Mycobacterium tuberculosis infection.

### Quorum Sensing

Without wishing to be bound by theory, quorum sensing is generally considered to represent a response to fluctuations in cell-population density. Quorum sensing microorganisms for instance bacteria, produce and release chemical signal molecules called autoinducers that increase in concentration as a function of cell density. The detection of a minimal threshold stimulatory concentration of an autoinducer leads to an alteration in gene expression. Microorganism such as Gram-positive and Gram-negative bacteria use quorum sensing communication circuits to regulate a diverse array of physiological activities. These processes include symbiosis, virulence, competence, conjugation, antibiotic production, motility, sporulation, and biofilm formation. In general, Gram-negative bacteria use acylated homoserine lactones as autoinducers, and Gram-positive bacteria use processed oligo-peptides to communicate. Recent advances in the field indicate that cell-cell communication via autoinducers occurs both within and between bacterial species.

A desirable level of antimicrobial metabolites can depend on the preferred subsequent uses of the antimicrobial composition.

In some embodiments the desirable level of antimicrobial metabolites is at the peak of log phase.

In some embodiments the growth or propagation of the antimicrobial culture is substantially reduced or stopped upon reaching the peak of log phase.

In some embodiments the antimicrobial metabolites can be toxic to pathogenic microorganisms such as bacterial pathogens. As used herein the term “toxic” refers to a toxin or other microbial substance or metabolite which induces an immune response especially the production of antibodies.

In various embodiments, the propagated antimicrobial culture can be useful for the treatment of mammalian in particular human diseases caused by microorganisms such as bacteria through the inhibition of the bacterial quorum sensing cascade rendering the pathogen avirulent. Such diseases include endocarditis, respiratory and pulmonary infections (preferably in immunocompromised patients), bacteraemia, skin conditions, vagina, colon, central nervous system infections, ear infections including external otitis, eye infections, bone and joint infections, urinary tract infections, gastrointestinal infections and skin and soft tissue infections including wound infections, pyoderma and atopic dermatitis which all can be triggered by Helicobacter spp such as Helicobacter pylori.

In some embodiments the propagated antimicrobial culture is lyophilised. As used herein the term “lyophilised” means preserving the propagated antimicrobial culture by freezing it very quickly and then subjecting it to a vacuum or sublimation to remove the ice. In some embodiments, the lyophilised antimicrobial culture is preserved long-term. In some embodiment the lyophilised antimicrobial culture comprises viable microbial cells. In some embodiment, lyophilisation can be used to prepare a dosage form that is to be reconstituted for injection. In some embodiments the propagated antimicrobial culture is dehydrates or otherwise dried.

In some embodiments the lyophilised antimicrobial culture is a lyophilised antimicrobial composition. In some embodiments the propagated antimicrobial culture is in the form of a powder.

In some embodiments the lyophilised antimicrobial culture is a lyophilised antibacterial composition. In some embodiments the lyophilised antibacterial composition comprises zelotite and at least one member of the Lactobacilli genus is Lactobacillus reuteri. In some embodiments, the lyophilised antimicrobial composition comprises a zelotite and Lactobacillus reuteri DSM17648. In some embodiments, the lyophilised antimicrobial composition comprises a zelotite, Lactobacillus reuteri DSM17648 and a mineral salt. In some embodiments, the lyophilised antimicrobial composition comprises a zelotite, Lactobacillus reuteri DSM17648, calcium carbonate and magnesium carbonate. In these embodiments the Lactobacillus reuteri strain may also be UBLRu-87.

In some embodiments there is provided a propagated antimicrobial culture combined with one or more pharmaceutically acceptable ingredients. In some embodiments there is provided a propagated antimicrobial culture combined with one or more pharmaceutically acceptable excipients or additive.

In the present context, a “pharmaceutically acceptable excipient” refers to an inert substance added to a pharmaceutical composition to further facilitate administration of the propagated antimicrobial culture, for example a lyophilised culture. Examples, without limitation, of excipients include calcium carbonate, calcium phosphate, various sugars and types of starch, cellulose derivatives, gelatine, vegetable oils and polyethylene glycols.

In some embodiments the pharmaceutical compositions comprise the antimicrobial agent. In some embodiments the pharmaceutical compositions comprised the antibacterial agent.

Thus, the present invention also relates to compositions including pharmaceutical compositions comprising a therapeutically effective amount of a propagated antimicrobial culture, for example a lyophilised culture, as mentioned herein. As used herein a propagated antimicrobial culture, for example a lyophilised culture, will be therapeutically effective if it is able to affect the target microorganism concentration.

Preferably, a propagated antimicrobial culture, for example a lyophilised culture or formulation, will be therapeutically effective if it is able to affect the target microorganism concentration where it is able to treat or prevent a microorganism related disease such as bacteria-related disease or disorder in a subject after the composition, for example a lyophilised composition, has been administered to a subject. In some embodiments the disease or disorder is associated with a pathogen.

In some embodiments, the pathogen is Helicobacter spp. In some embodiments the pathogen is selected from the group comprising Helicobacter canis, Helicobacter felis, Helicobacter heilmannii, Helicobacter mustelae, Helicobacter bizzozeronii, Helicobacter acinonyx, Helicobacter fenneliae, Helicobacter rappini, Helicobacter hepaticus, Helicobacter pullorum, Helicobacter bilis, Helicobacter rodentium, Helicobacter trogontum, Helicobacter cinaedi, Helicobacter muridarum, Helicobacter pametensis, Helicobacter cholecyctus and Helicobacter pylori.

In some embodiment of the present invention, the disease or condition is associated with or caused by Helicobacter canis.

In some embodiments, the pathogen is Helicobacter pylori.

In some embodiments, the pathogen is Staphylococcus spp. e.g. Staphylococcus capitius urealiticum infection; Proteus spp. e.g. Proteus mirabilis infection; Klebsiella spp. e.g. Klebsiella pneumoniae infection, and Mycobacterium spp. e.g. Mycobacterium tuberculosis infection.

In a further embodiment, the propagated antimicrobial culture, for example a lyophilised culture of the present invention can be administered directly to animals, preferably to mammals, and in particular to humans as non-antibiotic antibiotics per se, as mixtures with one another or in the form of pharmaceutical preparations which allow enteral or parenteral use and which as active constituent contain an effective dose of the propagated antimicrobial culture, for example a lyophilised culture, in addition to customary pharmaceutical excipients and additives.

As mentioned above, in addition to the propagated antimicrobial culture, the culture can contain further customary, usually inert carrier materials, additives or excipients. Thus, the culture can also contain additives or adjuvants commonly used for instance in galenic formulations, such as, e.g., fillers, extenders, disintegrants, binders, glidants, wetting agents, stabilizers, emulsifiers, preservatives, sweetening agents, colorants, flavourings or aromatisers, buffer substances, and furthermore solvents or solubilizers or agents for achieving a depot effect, as well as salts for modifying the osmotic pressure, coating agents or antioxidants. They can also contain two or more cultures and also other therapeutically active substances such as antivirals, antifungals or antibiotics.

Thus, the cultures of the present invention can be used alone, in combination with other compounds of this invention or in combination with other active compounds, for example with active ingredients already known for the treatment of the afore mentioned diseases, whereby in the latter case a favourable additive effect is noticed.

In another aspect, the present invention provides a process of preparing a product selected from food products, beverages, nutritional products, nutraceuticals and animal feed, the process comprising combining one or more ingredients with a propagated antimicrobial culture.

In some embodiment, the propagated antimicrobial culture products can incorporate inert, inorganic or organic excipients.

In some embodiments, to prepare pills, powders, tablets, coated tablets and hard gelatine capsules, e.g., lactose, corn starch or derivatives thereof, talc, stearic acid or its salts, etc. can be used. Excipients for soft gelatine capsules and suppositories are, e.g., fats, waxes, semi-solid and liquid polyols, natural or hardened oils etc. Suitable excipients for the production of solutions and syrups are, e.g., water, alcohol, sucrose, invert sugar, glucose, polyols etc.

MATERIALS AND MRETHODS

1. Growth and Laboratory Maintenance of H. pylori

Different microbiological techniques and methods of maintaining and growing H. pylori would be known to the skilled person.

An example of such laboratory techniques which have been employed in the present invention can be found in following link:

https://www.ncbi.nlm.nih.gov/pmc/articles/
PMC3357201/# :~: text=CULTURE%20OF
%20HELICOBACTER%20ORGANISMS%20ON,
as%20horse%2C%20ox%2C%20or%20sheep

### 2. Test for Urease Output

Urease activity assay kit (Sigma Aldrich)

The urease test identifies those organisms that are capable of hydrolyzing urea to produce ammonia and carbon dioxide. It is primarily used to distinguish urease-positive Proteeae from other Enterobacteriaceae.

### Method 1

The ureolytic activity assay with the M9U medium is capable of screening cell-based and cell-free urease activity. Urea-containing growth medium and method for continuous real-time monitoring and screening of urease activity from both bacterial cells and pure urease in a plate reader setup. The defined M9-based urea (M9U) medium was found to be more sensitive and suitable for a plate reader setup than both Christensen's urea broth (CUB) and Stuart's urea broth (SUB), which are established and well-known complex urea media that formed the principle foundation of M9U. Furthermore, the urease activity measurements using the M9U medium in our plate reader-based method allow reliable high-throughput screening of urease inhibitors.

Development of a M9-based urea medium (M9U) for sensitive and real-time monitoring of ureolytic activity of bacteria and cell-free urease Jens Jakob Sigurdarson, Simon Svane, Henrik Karring:https://doi.org/10.1002/mbo3.976

### Method 2

The developed assay is a robust cellular model to test, directly in the cell environment, urease inhibitors. The efficacy of a co-expressed peptide to affect the interaction between UreF and UreD, two accessory proteins necessary for urease activation, was observed. This event involves a process that occurs through folding upon binding, pointing to the importance of intrinsically disordered hot spots in protein interfaces.

Targeting Helicobacter pylori urease activity and maturation: In-cell high-throughput approach for drug discovery CinziaTarsia, AlbertoDanielli, FrancescaFlorini, PaoloCinelli, StefanoCiurli, Barbara Zambelli.

Two media types are commonly used to detect urease activity.

Christensen's urea agar is used to detect urease activity in a variety of microorganisms. Stuart's urea broth is used primarily for the differentiation of Proteus species. For Christensen's urea agar, urease production is indicated by a bright pink (fuchsia) color on the slant that may extend into the butt after 1-6 hours of incubation.

The culture medium will remain a yellowish colour if the organism is urease negative.

For Stuart's urea broth, urease production is indicated by a bright pink (fuchsia) colour throughout the broth.

TEST FOR AMMONIA—EXAMPLE PREDICTIVE MARKER

Ammonium test kit Quantofix (Sigma Aldrich) Ammonia Assay Kit (Sigma Aldrich).

Four solutions with the following concentrations: 50, 100, 300, and 500 mg L−1 N—NH4+were prepared from the standard solution of ammonium chloride at concentration of 1 g L−1 These solutions were subjected to the pH analysis performed with the potentiometric method.

Samples of 1, 2 g & 3 g of clinoptilolite were weighed and 100 ml of N—NH4+ solutions were added. The prepared samples were shaken for 30, 60 and 180 minutes and after that the pH was measured. Next, the samples were filtered in order to determine the concentration of ammoniacal nitrogen.

Each solution was prepared in triplicates.

The ammonium concentration of the aqueous phase was determined by the standard distillation method. Prior to the determination of ammoniacal nitrogen the filtered samples were subjected to distillation (BÜCHI 323) and the concentration of N—NH4 + was determined using spectrophotometer (Hach).

TEST FOR ANTIMICROBIAL COMPOSITIONS A, B, C & D REDUCTION OF AMMONIA, AMMONIUM AND UREASE (EXAMPLE PREDICTIVE MARKERS)

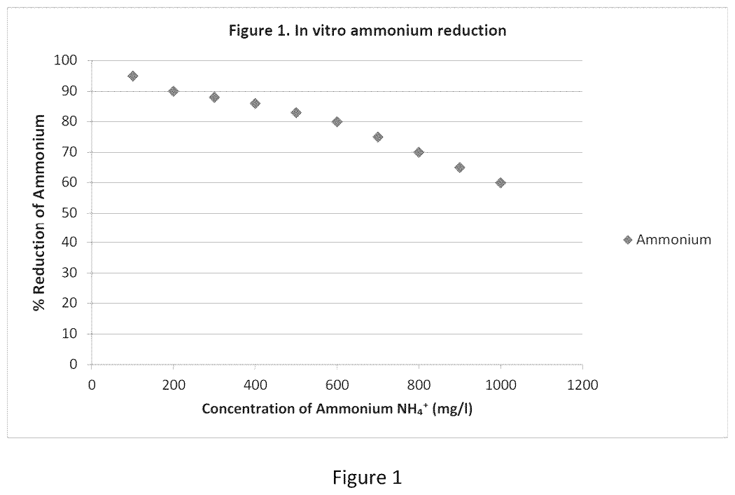

In FIG. 1 there is described an in vitro urea and ammonia reduction assays data using the antimicrobial compositions of the invention.

Fixed Bed Ion Exchange Column Studies In order to test the feasibility of natural ZeoliteStilbite as an adsorbent for the removal of nitrate from groundwater, a continuous mode of application was studied in a glass column (60 cm×0.8 cm).

Natural ZeoliteStilbite was suspended in distilled water for about 10 minutes and was then used for column studies. glass wool was kept at the bottom of the column to avoid the losses of sorbent with the flow of nitrate solution. Then the sorbents was transferred onto the glass wool in the column. Nitrate solution was fed into the column at the flow rate of 10 ml/min using a peristaltic pump. Journal of Water Resource and Hydraulic Engineering December 2014, Vol. b, Iss. 4, PP. 74-80-77-determine exhaustive capacity, 50-ml fractions of the effluent were collected from the bottom of the column.

The process was continued until the amount of nitrate in the effluent was the same as that in the feed. PH was measured using pH meter (Elico Instruments model LI-107). The effect of pH was studied in the pH range 2 to 7 by adjusting pH of nitrate solutions using dilute HCL and NaOH solutions.

The effect of flow rate was studied with different flow rate between 1 to 5 mL per min. by using peristaltic pump. The effect of different initial concentration was studied with different concentration nitrate solution in the range between 40 to 240 mg/L.

After each service cycle of the exchange, a regeneration cycle took place with the solution of sodium nitrate with the concentration of 30 g/L, by passing the solution from the top of the column through the fixed bed of Zeolite. The regeneration process was stopped when the nitrate ion concentration in the effluent became.

TEST FOR THE ABILITY OF ANTIMICROBIAL COMPOSITIONS A, B, C & D, TO EFFECTIVELY REDUCTION OF H. PYLORI LOAD

MODEL GUT TESTS WITH HIGH AMMONIA/AMMONIUM, WHAT EFFECT DOES EACH ONE OF ANTIMICROBIAL COMPOSITIONS A, B, C & D HAVE?

AMMONIA EXCHANGE CAPACITY (AEC), OPTIMISATION

The inhibitory effect of Ca2+ on NH4+ exchange efficiency was fitted to the competitive inhibition Monod model with half-saturation rate constant of 134.7 mg L-1. Ca2+ addition reduced the NH4+ removal rate and lengthened the exchange equilibrium time of zeolite.

Periodic precipitation of Ca2+ in the form of calcium carbonate from the used regenerant maintained the removal efficiency of NH4+ commendably by alleviating inhibition effect of Ca2+ and extended the working life of zeolite.

Compared to Bohart-Adams model and Thomas model, the Dose-Response model could predict the breakthrough curve well, and the fitted parameter further confirmed that NaClO—NaCl regeneration with periodic Ca2+ removal is an effective method to maintain efficient NH4+ from wastewater by zeolite.

### EXAMPLES

It is known that H. pylori infection is associated with elevated levels of ammonia and/or urea which allow members of the pathogen to survive and thrive in a higher pH as may be found in the GIT.

However, removing or reducing the levels of ammonia and/or urea may adversely affect the survival of H. pylori if exposed to physiologically normal gastric acid conditions. The ability of the antimicrobial composition of the present invention to remove or reduce ammonia and/or urea levels that Helicobacter spp,. for example, Helicobacter pylori produce was tested.

Lactobacillus L. reuteri a Gram positive (+ve) bacteria aggregates to the exposed H. pylori and with the active mineral salts such as calcium carbonate and/or magnesium carbonate help remove the pathogen from the subject.

The carbonates help to ease stomach discomfort and contribute to rebalance the natural action of the stomach.

Lactobacillius L. reuteri can survive the normal gastric acid conditions of the stomach populating the microbiota much more favourably.

If a subject suffers with the following symptoms of H. pylori: bloating, abdominal pain worse when your stomach is empty, nausea, shortness of breath, acid reflux, high ammonia/ammonium levels, ulcers, gastritis, gastric cancer, black tarry stool.

Example—antimicrobial composition A used in to treat H. pylori pathogen infection:
3.5 g double activated Zeolite Clinoptilolite
10 billion viable organisms L. reuteri DSM17648 culture.

Example—antimicrobial composition B used in tests to treat H. pylori pathogen infection:
2 g calcium carbonate
2 g magnesium carbonate
3.5g double activated Zeolite Clinoptilolite
10 billion viable organisms L. reuteri DSM17648 culture.

Example—antimicrobial composition C for treating acid reflux and bloating associated with H. pylori infection.
1.5 g double activated Zeolite Clinoptilolite
5 billion viable organisms L. reuteri DSM17648 culture.

Example-antimicrobial composition D for treating acid reflux and bloating associated with H. pylori infection:
1 g calcium carbonate
1 g magnesium carbonate
1.5 g double activated Zeolite Clinoptilolite
5 billion viable organisms L. reuteri DSM17648 culture.

Antimicrobial composition A, B, C or D can be employed in a regimen for treating H. pylori pathogen infection in a subject such as a human.
Sachet of powder such as lyophilised culture of the antimicrobial composition of choice-dissolved in water;
Liquid Suspension in Lactulose
Tablet format

Antimicrobial composition A, B, C or D employed in a regimen for treating H. pylori pathogen infection in a subject such as an animal.
Sachet of powder such as lyophilised culture of the antimicrobial composition of choice-dissolved in water;
Liquid suspension in lactulose

Composition and Physicochemical Properties of the Tribomechanically Activated Zeolite Clinoptilolite (TMAZ®)

Chemical composition SiO2, 65.0-71.3%; Al2O3, 11.5-13.1%; CaO 2.7-5.2%; K2O, 2.2-3.4%; Fe2O3, 0.7-1.9%; MgO, 0.6-1.2%; Na2O, 0.2-1.3%; TiO2, 0.1-0.3%; Si/Al ratio, 4.8-5.4.

Empirical formula (Ca, K2,Na2,Mg)4Al8Si40O96×24H2O
Physicomechanical properties Specific mass, 2.2-2.5 g/cm3; porosity, 32-40%; effective pore diameter, 0.4 nm
Ion-exchanging capacity Total exchange capacity, 1.2-1.5 mol/kg; Ca2+, 0.64-0.98 mol/kg; Mg2+, 0.06-0.19 mol/kg; K+, 0.22-0.45 mol/kg; Na+, 0.01-0.19 mol/kg Ion-exchanging selectivity Cs>NH4+>Pb2+>K+>Na+>Mg2+>Ba2+>Cu2+>Zn2+

Chemicals absorbed NH3, hydrocarbons C1-C4, CO2, H2S, SO2, NOX, aldehydes.
Toxicity Nontoxic; generally recognized as safe (GRAS) according to US Code of Federal Regulations (21 CFR 182, Subpart C).

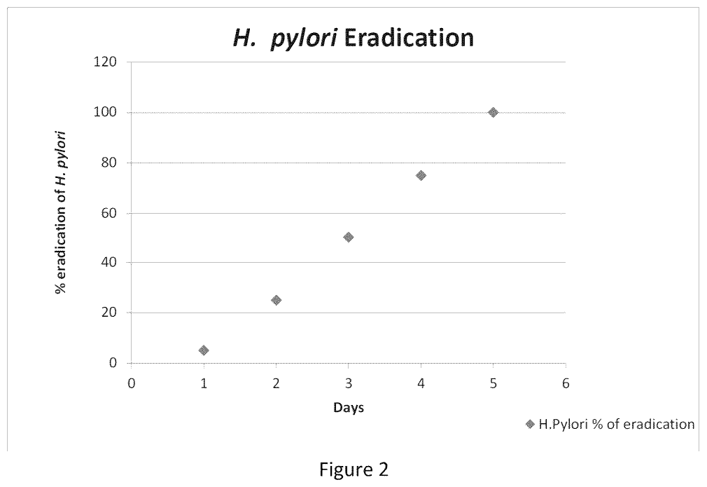

Patient effective regimen for administration for H. pylori—duration of regimen 5 days—FIG. 2—Showing cfu before treatment and after treatment.
Day 1-5
No food prior to 8am treatment
8 am Antimicrobial Composition of choice (here we use antimicrobial composition B) in 500 ml water, taken on an empty stomach
9 am dry toast, drink lots of water
10 am Antimicrobial Composition B in 500 ml water
11 am dry toast, drink lots of water
12 pm Antimicrobial Composition B in 500 ml water
After 1pm eat and drink as normal.

Effective in vivo testing on a subject such as a human patient in a clinical setting.

Prior to May 2020 the patient was a healthy, active 45 year old women and mum of 3 children.

No previous symptoms of stomach issues and a consistent flat stomach that returned to normal shortly after all 3 child births.

In May 2020 the patient had severe bloating of the stomach and the patient visited the GP. The GP informed the patient that the symptoms were simply age and hormone related with no tests were necessary just a hand examination of the abdominal area.

However, the patient's vision started to deteriorate, joints started to stiffen and ache and the bloating of the stomach persisted and worsened pushing up against my lungs making me breathless.

In 1 Jul. 2021 an x-ray confirmed mild to moderate degeneration of metatarsophalangeal joint in the patient's right foot.

Blood tests were taken, 29 Jun. 2021 Serum vitamin B12 level (XE2pf) 107 ng/L (150-1000) was reported abnormally low and the patient had mild hypothyroidism.

A series of no less than six (6) B12 injections were administered as a loading dose within 2 weeks 12th-26th July.

Following the identification of an H. pylori infection in a stool test on the 26 Jul. 2021, the patient was prescribed the NHS protocol triple therapy; amoxicillin 500 mg, Clarithromycin 500 mg, lansoprazole 30 mg, to be taken for 7days.

The patient completed the prescribed standard of care for treatment of H. pylori infection antibiotic course and re-tested the stool sample in September 2021. However, the test results remained positive with no discernible improvement of the symptoms.

The patient was treated using with combined calcium carbonate, magnesium carbonate, Zeolite Clinoptilolite and L. reuteri for 5 days (antimicrobial composition B as detailed in Section. Patient effective regimen or course for administration and treatment for H. pylori—duration of regimen 5 days).

The patient completed this course and re-tested the stool sample 8 Oct. 2021, the test results were negative.

30th November Serum thyroid-stimulating hormone (TSH) level 5.6 mlU/L (0.3-4.5) hypo thyroidism result abnormal—before treatment.

31st January Serum TSH level 4.0 6mlU/L (0.3-4.5) result normal—after treatment using the antimicrobial compositions of the present invention.

It is hypothesised that the body of the subject such as a human naturally creates more acid to combat Helicobacter spp such as H. pylori in an inflammatory response. The body also recognises that when the acid levels become too high it needs buffering by deploying the assistance of stores of calcium and magnesium to achieve this. However, over time, for instance, weeks, months or ever years of persistent Helicobacter spp such as H. pylori infection, too much acid and depleted stores of magnesium and calcium may lead to other ailments and concomitant symptoms, alongside the gastric disorders potentially caused by the auto immune response and colonisation from the pathogen. Using the non-antibiotic antibacterial compositions of the present invention we were not only able to effectively reduce ammonia and urea levels but also in vivo in a patient eliminate H. pylori but are able to prevent associated symptoms, diseases or conditions which are linked to or associated with Helicobacter spp such as H. pylori infection. Similar results are expected with Lactobacillus reuteri UBLRu-87.

Further Experimental Work

Additional experimental work was carried out to determine the viability of H. pylori at varying pH levels.

Work was carried out to culture strains of H. Pylori.

All test organisms were cultured on Brain Heart Infusion agar (BHIA)+5% blood (blood should be approx. 2 weeks old) and incubated at 37° C. for up to 7 days in the DG250 microaerophilic cabinet supplied with 10% CO2, 5% O2 and 85% N2.

An inoculum equivalent to a 2 McFarland (1-10×107 CFU/mL) was prepared in 5 mL sterile phosphate-buffered saline (PBS) for each test strain. Each inoculum suspension was diluted 1:10 (2 mL inoculum +18 mL media) or 1:100 (200 μL+19.8 mL media) into each growth medium +10% [v/v] foetal bovine serum (FBS). A media only (sterility) control was also included.

Immediately following inoculation, a 0.5 mL sample was taken and placed in a sterile Eppendorf tube. From the 1 mL sample total viable count was determined using Miles and Misra methodology on brain heart infusion (BHI) agar containing 5% blood. Plates were incubated for ˜5 days in microaerophilic conditions until suitable growth could be quantified. Ammonium levels were also detected by adding 2 drops of test reagent to 1 mL of sample and using the dip stick to quantify the levels present in the samples (Quantofix lot 315250)

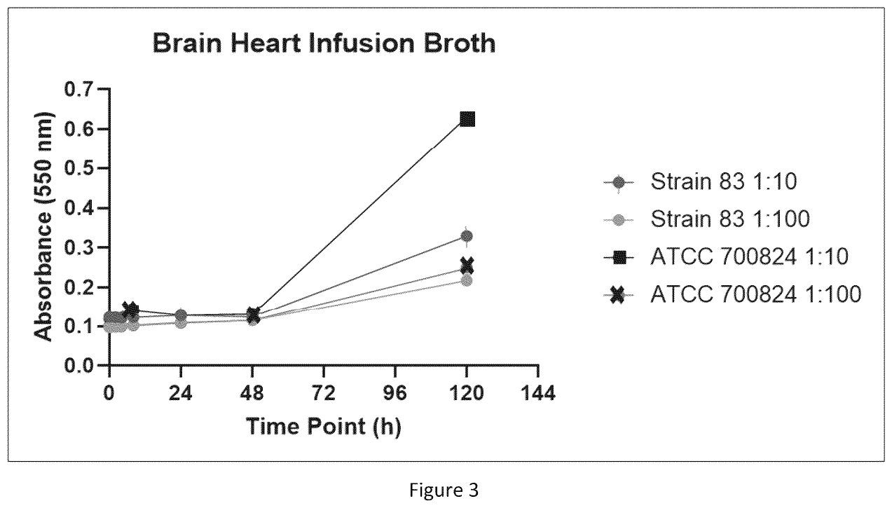

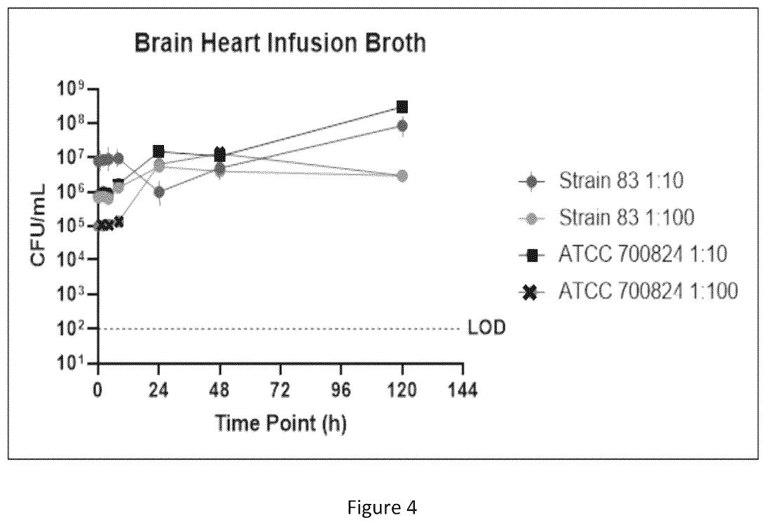

From the remaining sample, 200 μL was removed to a sterile 96-well flat bottom microdilution plate (Corning; #3370) and absorbance at a wavelength of 550 nm recorded in a plate reader. Sampling was repeated at 2, 4, 8, 24, 48 and 124 hours post inoculation. The results are shown in FIG. 3 (blank adjusted absorbance of H. Pylori Strain 83 and ATCC 700824) and FIG. 4 (Total viable count of H. pylori Strain 83 and ATCC 700824).

The effect of pH on total viable cell count (TVC) and ammonium concentration was investigated.

H. pylori ATCC 700824 was cultured on Brain Heart Infusion (BHI)+5% blood (blood should be approx. 2 weeks old) and incubated at 37° C. for up to 7 days in the DG250 microaerophilic cabinet supplied with 10% CO2, 5% O2 and 85% N2.

An inoculum equivalent to a 2 McFarland (1-10×107 CFU/mL) was prepared in sterile phosphate-buffered saline (PBS) and diluted 1:10 (20 mL inoculum+180 mL media) into BHI +10% [v/v] foetal bovine serum (FBS) and 0.1% [w/v] yeast extract and incubated for 96 h in microaerophilic conditions.

The culture was split into 9 20mL aliquots and centrifuged at 3500rpm for 10 minutes and the supernatant removed. The cell pellet was re-suspended in 20 mL buffered BHI +10% [v/v] foetal bovine serum (FBS) and 0.1% [w/v] yeast extract adjusted to pH 2, 5 or 7 with 5 M hydrochloric acid supplemented with 0, 10 or 50 mM urea to make a total of 9 samples to test, (plus sterility control at each urea concentration.)

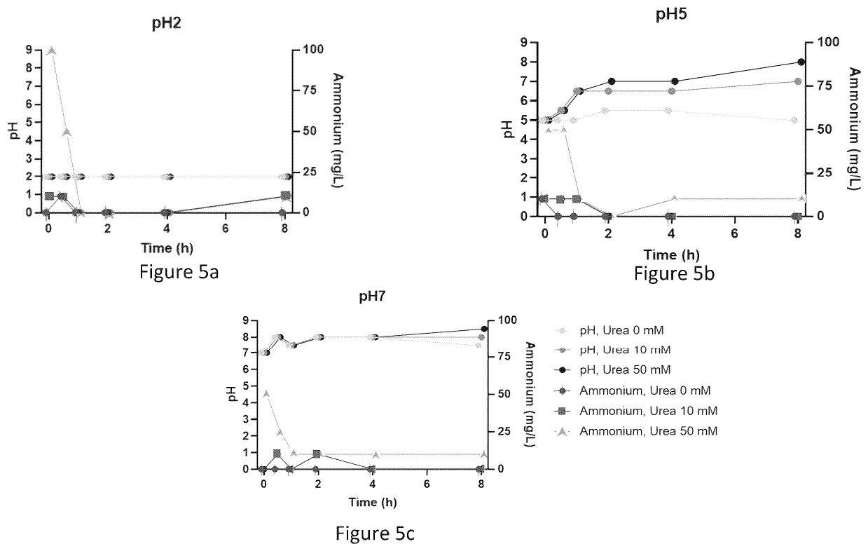

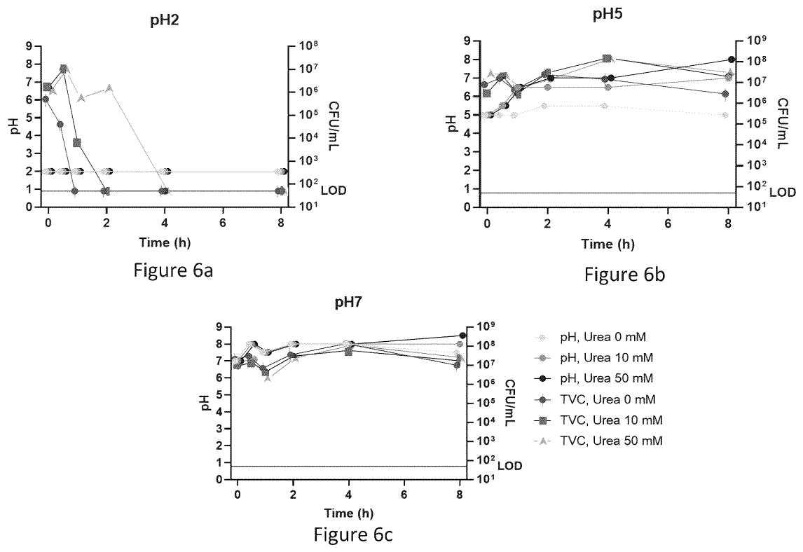

Samples were removed at 0, 0.5, 1, 2, 4 and 8 h post inoculation and total viable count determined using Miles and Misra methodology on BHI agar containing 5% blood. The results can be seen in FIG. 5. Plates were incubated for ˜5 days in microaerophilic conditions until suitable growth could be quantified. Ammonium concentrations (Quantofix lot #315250) and pH (Fisherbrand lot #18F0131) were determined using semi-quantitative colorimetric strips. The results can be seen in FIG. 6.

This work confirmed that H. pylori cannot not survive in an acidic gastro intestinal tract without some mechanism of survival. Without wishing to be bound by theory, it is hypothesised that H. pylori survives in the gastro-intestinal tract, and particularly in the stomach, in a periplasm (see Bury-Moné, S., Skouloubris, S., Labigne, A. and De Reuse, H. (2001), The Helicobacter pylori UreI protein: role in adaptation to acidity and identification of residues essential for its activity and for acid activation. Molecular Microbiology, 42:1021-1034 and Wen Y, Scott DR, Vagin O, Tokhtaeva E, Marcus EA, Sachs G. Measurement of Internal pH in Helicobacter pylori by Using Green Fluorescent Protein Fluorimetry. J Bacteriol. 2018 June 25; 200(14): e00178-18. doi:10.1128/JB.00178-18. PMID: 29735759; PMCID: PMC6018362. ). The periplasm acts to allow the bacterium to adapts the pH in its own environment to survive through hydrolysis of urea. The ammonium output resulting from this hydrolysis of urea calibrates the pH creating optimum conditions for the survival of H. pylori. Other research has also illustrated this mechanism of survival, which differs to the majority of literature which has suggested that H. pylori survives in the gastrointestinal tract hidden under the mucus layer. The inventor here hypothesises that both schools of thought are correct. As such they propose a double action treatment to eradicate the bacteria from the GI tract. L. reuteri (or a similar Lactobacilli) binds to the bacteria which helps to bring it out of the mucus lining of the gastrointestinal tract, however, this does not necessarily remove it from the body efficiently. A zeolite such as clinoptilolite or Na-clinoptilolite absorbs the H. pylori & L. reuteri (or other lactobacilli). It is hypothesised that the cation absorption capacity of the zeolite e.g. clinoptilolite, exchanges sodium, calcium and/or magnesium ions for ammonium. The presently proposed composition of zeolite and at least one member of the Lactobacilli genus is insoluble and as such is efficiently removed from the body taking the H. pylori with it. The presently proposed composition of zeolite and at least one member of the Lactobacilli genus thus provides a unique, synergistic and effective treatment for H. pylori that is a non-antibiotic treatment created that can be used to efficiently eradicate H. Pylori infections, urease positive bacteria & ammonium in the gastrointestinal tract.

Initial modelling suggests that the presently proposed composition of zeolite, in particular where the zeolite is activated clinoptilolite, and at least one member of the Lactobacilli genus, such as L. reuteri, allows for more rapid removal of H. pylori than with zeolite alone. Initial modelling suggests that the presently proposed composition of zeolite and at least one member of the Lactobacilli genus allows for more complete removal of H. pylori from a patient's gastrointestinal tract than with zeolite alone.

It is thought that as least part of the composition is substantially water insoluble at body temperature. It is likely that at least a substantial portion or all of the the zeolite component is insoluble, and that this assists with the clearance.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. An antimicrobial composition comprising a zeolite and at least one member of the Lactobacilli genus, for use in a treatment of medical conditions associated with a pathogenic urease active bacterial infection in the gastro-intestinal tract in vertebrates.

2. (canceled)

3. (canceled)

4. The antimicrobial composition of claim 1, wherein the zeolite is zeolite clinoptilolite (ZC).

5. The antimicrobial composition of claim 1, wherein the ZC is activated.

6. The antimicrobial composition of claim 1, wherein the at least one member of the Lactobacilli genus is selected from the group consisting of Lactobacillus reuteri, Lactobacillus delbrueckii, Lactobacillus rhamnosus, Lactobacillus casei, Lactobacillus acidophilus, Lacticaseibacillus paracasei, Lactiplantibacillus plantarum, Levilactobacillus brevis, Ligilactobacillus salivarius, Limosilactobacillus fermentum, Lactobacillus bulgaricus, Lactobacillus crispatus, Lactobacillus helveticus and Lactobacillus johnsonii.

7. (canceled)

8. The antimicrobial composition of claim 6, wherein the Lactobacillus reuteri is Lactobacillus reuteri DSM17648 or Lactobacillus reuteri UBLRu-87.

9. (canceled)

10. (canceled)

11. (canceled)

12. (canceled)

13. The antimicrobial composition of claim 1, wherein the antimicrobial composition further comprises biologically active substances or minerals such as calcium carbonate, magnesium carbonate, vitamins, such as D and E, pharmaceutically acceptable carriers, additives and adjuvants, as well as antimicrobial chitin, such as chitosan and alpha-ketoglutarate, citrate and lactate.

14. (canceled)

15. The antimicrobial composition of claim 1, preceding composition comprising activated zeolite clinoptilolite (aZC), Lactobacillus reuteri DSM17648, calcium carbonate and magnesium carbonate.

16. The antimicrobial composition of claim 1 claims, wherein the infection is associated with the group consisting of Helicobacter pylori, Staphylococcus species (spp.), Proteus spp., Klebsiella spp., and Mycobacterium spp.

17. (canceled)

18. (canceled)

19. The antimicrobial composition of claim 1, wherein the antimicrobial composition leads to a reduction of one or more markers or predictive marker selected from the group consisting of urea, gastric ammonia, acid reflux, bloating and urease activity.

20. (canceled)

21. (canceled)

22. (canceled)

23. The antimicrobial composition of claim 19, wherein the reduction of gastric ammonia is at least 5% compared to placebo.

24. The antimicrobial composition of claim 19, wherein the reduction of gastric ammonia is at least 0.1 compared to placebo.

25. (canceled)

26. The antimicrobial composition of claim 1, wherein the antimicrobial composition leads to a statistically significant clinical improvement in a human infected with one or more of Helicobacter pylori, Staphylococcus spp., Proteus spp., Klebsiella spp., and Mycobacterium spp.

27. The antimicrobial composition according to claim 23, wherein the reduction of gastric ammonia marker levels in a human infected with Helicobacter pylori is statistically significant.

28. The antimicrobial composition of claim 1, wherein the antimicrobial composition leads to a statistically significant clinical improvement in a human infected with Helicobacter pylori.

29. The antimicrobial composition of claim 1, wherein the antimicrobial composition is substantially insoluble in water.

30. A method of treatment, alleviation or prophylaxis of Gastrointestinal Tract (GIT) disease or disorder, gastritis, gastric ulcer, duodenal ulcer, gastric cancer, duodenal cancer in vertebrates, including mammals and birds, in need of such treatment, alleviation or prophylaxis, the method comprising administering by separate, combined or concomitant administration of a pharmaceutical formulation comprising the antimicrobial composition according to claim 1.

31. (canceled)

32. The method according to claim 30, wherein the pharmaceutical formulation in administered as one, two, three, four or more doses or sub-doses per day or per administration.

33. The method according to claim 30, wherein the pharmaceutical formulation comprises from 10⁷ to 10⁹ CFU/g of Lactobacillus reuteri DSM17648 and zeolite, wherein the zeolite is present in an amount of at least 50 mg/gram of the pharmaceutical formulation.

34. The method of claim 33, wherein the pharmaceutical formulation further comprises a mineral salt such as calcium carbonate and/or magnesium carbonate, wherein the calcium carbonate and magnesium carbonate are each present in an amount of at least 0.5 mg/gram of the formulation.

35. (canceled)

36. The method of claim 30, wherein the treatment leads to complete remission of the disease or disorder caused by or associated with Helicobacter spp. infection.

37. (canceled)

38. (canceled)
